SEQUENCE LISTING (1 sequence)

SEQ ID NO: 1 - protein, 9 aa, Unknown
  Description of Unknown bradykinin peptide
RPPGFSPFR

(19) United States
(12) Patent Application Publication
(10) Pub. No.: US 2016/0030416 A1
(43) Pub. Date: Feb. 4, 2016

(54) METHODS OF TREATING B2-BRADYKININ RECEPTOR MEDIATED ANGIOEDEMA

(71) Applicant: SHIRE HUMAN GENETIC THERAPIES, INC., Lexington, MA (US)

(72) Inventors: Kevin Leach, Wellesley, MA (US); Teresa Wright, Lexington, MA (US); Brian Felice, Wellesley, MA (US); Richard Pfeifer, North Granby, CT (US); Pericles Calias, Melrose, MA (US); Thomas McCauley, Cambridge, MA (US)

(21) Appl. No.: 14/776,542

(22) PCT Filed: Mar. 12, 2014
(86) PCT No.: PCT/US14/24540

§ 371 Date: Sep. 14, 2015

Related U.S. Application Data
(60) Provisional application No. 61/786,126, filed on Mar. 14, 2013.

(51) Int. Cl. A61K 31/4709; A61K 9/00; A61K 31/185; A61K 9/10; A61K 31/47; A61K 45/06; A61K 47/38

(52) U.S. Cl. CPC A61K 31/4709; A61K 45/06; A61K 9/0053; A61K 47/38; A61K 9/10; A61K 31/47; A61K 31/185

(57) ABSTRACT

Methods of treating B₂-bradykinin receptor mediated angioedema in a subject by administering a composition containing a 8-(heteroarylmethoxy)quinolone compound, a 8-(arylmethoxy)quinoline compound, or a salt, a stereoisomer, a hydrate, or a solvate thereof. Oral formulations containing a 8-(heteroarylmethoxy)quinolone compound, a 8-(arylmethoxy)quinoline compound, or a salt, a stereoisomer, a hydrate, or a solvate thereof for the treatment of B₂-bradykinin receptor mediated angioedema. Use of a composition containing a 8-(heteroarylmethoxy)quinolone compound, a 8-(arylmethoxy)quinoline compound, or a salt, a stereoisomer, a hydrate, or a solvate thereof for the manufacture of a medicament for the treatment and/or prevention of a B₂-bradykinin receptor mediated angioedema.

### CROSS REFERENCE TO RELATED APPLICATIONS

This application claims the benefit of, and relies on the filing date of, U.S. provisional patent application No. 61/786,126, filed 14 Mar. 2013, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Mar. 11, 2014, is named 0138.0001-PCT_SL.txt and is 494 bytes in size.

### BACKGROUND

Hereditary angioedema (HAE) is a rare and potentially life-threatening genetic condition. HAE symptoms include episodes of edema (swelling) in various body parts including the hands, feet, face, intestinal walls and airways. Most HAE patients (those with Type I and Type II HAE) have a defect in the gene that controls the blood protein, C1 esterase inhibitor (C1-INH). The genetic defect results in production of either inadequate (Type I HAE) or non-functioning (Type II HAE) C1-INH protein. The genetic defects related to C1-inhibitor that cause Type I and Type II HAE are autosomal dominant. However, absence of a family history of HAE does not rule out an HAE diagnosis. It has been reported that as many as 20% of HAE cases result from patients who had a spontaneous mutation of the C1-inhibitor gene at conception.

Normal C1-INH protein helps to regulate the complex biochemical interactions of blood-based systems involved in disease fighting, inflammatory response and coagulation. Because defective C1-INH protein does not adequately perform its regulatory function, a biochemical imbalance can occur and produce unwanted peptides that induce the capillaries to release fluids into surrounding tissues, thereby causing edema.

Most attacks of HAE occur spontaneously, although anxiety, stress, minor trauma, surgery and illness have been cited as triggers. Untreated, an average HAE attack lasts twenty-four to seventy-two hours, but some residual swelling can persist for up to three or more days. Swelling of the extremities can be painful and debilitating depending on the location of the edema. Attacks that involve the face and/or throat are considered to be a medical emergency, because swelling of the throat can close the airway and lead to death by asphyxiation. Abdominal attacks cause severe pain, nausea, vomiting, dehydration and watery diarrhea. Further, abdominal attacks can mimic a surgical abdomen and many patients have been subjected to unnecessary exploratory surgery.

Deficiency of C1-inhibitor permits plasma kallikrein activation, which leads to the production of the vasoactive peptide bradykinin. Bradykinin (BK) is a vasoactive nonapeptide, H-Arg-Pro-Pro-Gly-Phe-Ser-Pro-Phe-Arg-OH (SEQ ID NO:1), formed locally in tissues, often in response to a trauma. Two types of BK receptors are recognized in mammals, B1 and B2. The actions of BK mediated by the B₂-bradykinin receptor are important physiological functions, such as the increase of vascular permeability, modulation of inflammatory responses and pain, and induction of vasoactive effects (vasodilatation, vasoconstriction). Surplus bradykinin results in inflammation, such as swelling, redness, overheating, and pain.

Bradykinin is responsible for the clinical symptoms of HAE, causing increased vascular permeability, vasodilation, and contraction of visceral smooth muscle. Thus, after an inciting factor, a quantitative or qualitative deficiency of C1-INH leads to inadequate regulation of bradykinin production and increased vascular permeability. Extravasation of fluid leads to non-pruritic edema. As high molecular weight kininogen is exhausted and bradykinin degraded, the edema begins to subside and the fluid is resorbed by the lymphatic system.

Peptide and non-peptide antagonists of B₂-bradykinin receptor have been described in the art. Firazyr® (injected icatibant) is a peptidomimetic drug consisting of ten amino acids that is a selective and specific antagonist of B₂-bradykinin receptor and has been used to treat acute attacks of HAE in adults with C1-esterase inhibitor deficiency. Ecallantide (trade name Kalbitor®, investigational name DX-88) is a drug used for the treatment of acute attacks of HAE. It is an inhibitor of the protein kallikrein and a 60-amino acid polypeptide. Also purified (C1INHRP) or recombinant (rhC1INH) human C1-inhibitor has been used in the treatment of acute attacks of HAE.

There are drawbacks with existing treatments. C1-inhibitor replacement products must be reconstituted prior to use and are administered intravenously. Prophylactic therapy with C1-inhibitor products requires intravenous administration twice weekly and only prevents ˜50% of attacks. Androgens are used for prophylaxis, but there are long-term side effects and they are not recommended for female and pediatric patients. Ecallantide, a subcutaneous (SC) treatment for acute HAE attacks, has a documented risk of anaphylaxis and must be administered by a healthcare professional in a hospital setting. Icatibant, which has been approved in the U.S. for subcutaneous self-administration during acute attacks of HAE, produces injection site reactions.

Methods of treating B₂-bradykinin receptor mediated angioedema are desirable. Treatment methods using small molecule B₂-bradykinin receptor antagonists are of interest. Also oral therapies for treating B₂-bradykinin receptor mediated angioedema are desirable.

### SUMMARY

Certain embodiments are drawn to methods of treating a B₂-bradykinin receptor mediated angioedema in a subject comprising administering to the subject in need thereof a therapeutically effective amount of a composition comprising a compound having formula (I) or a pharmaceutically acceptable salt, stereoisomer, hydrate, or solvate thereof, wherein plasma extravasation in the subject is reduced upon administration of the compound or the pharmaceutically acceptable salt, stereoisomer, hydrate, or solvate thereof and formula I is as follows:

wherein R₁ is

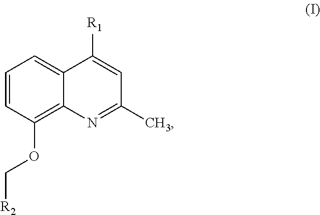

### H or

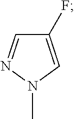

wherein R₂ is

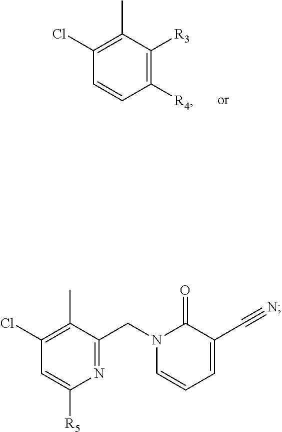

wherein R₃ is Cl or CN;
wherein R₄ is

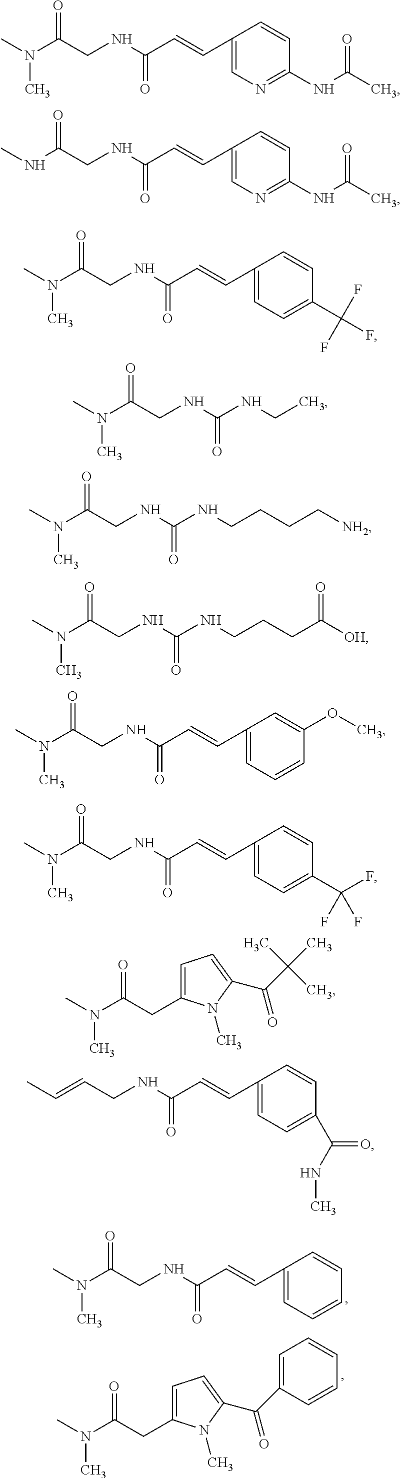

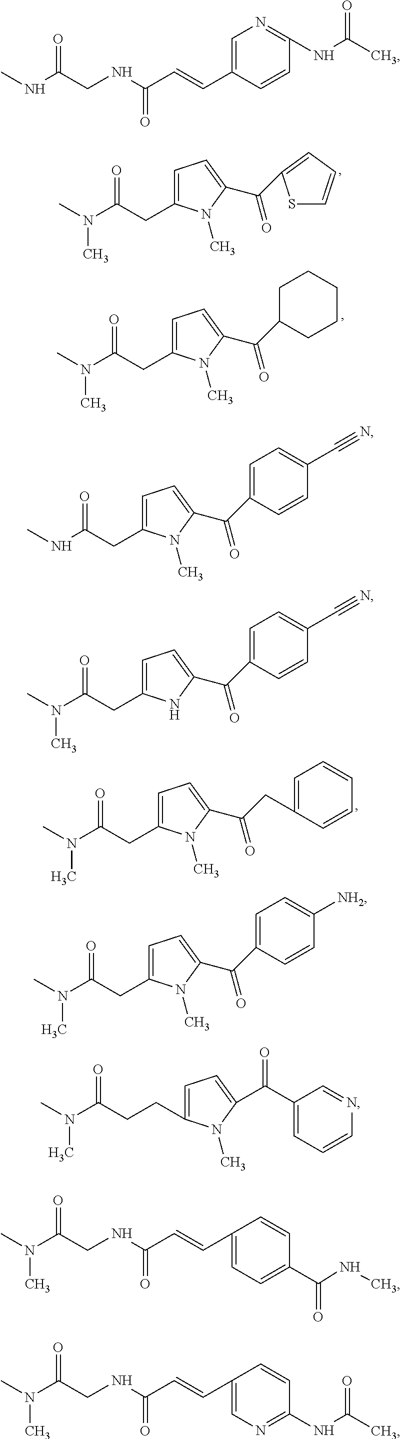

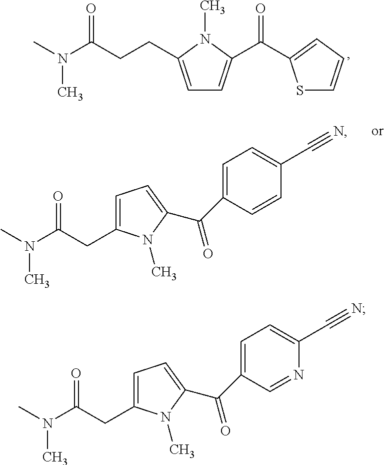

wherein R₅ is selected from the group consisting of H, a methyl group, an ethyl group, a propyl group, a butyl group, a pentyl group, or a hexyl group.

Some embodiments are drawn to methods of treating a B₂-bradykinin receptor mediated angioedema in a subject comprising:
administering to the subject in need thereof a therapeutically effective amount a composition comprising a compound having formula (II) or a pharmaceutically acceptable salt, stereoisomer, hydrate, or solvate thereof

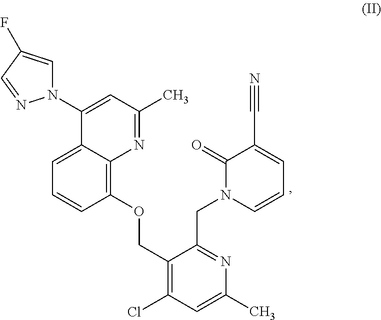 [SMILES: Cc1cc(Cl)c(COc2cccc3c(-n4cc(F)cn4)cc(C)nc23)c(Cn2cccc(C#N)c2=O)n1]

thereby reducing plasma extravasation in the subject.

Embodiments are drawn to methods of treating a B₂-bradykinin receptor mediated angioedema in a subject comprising:

administering to the subject in need thereof a therapeutically effective amount a composition comprising
11-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile;
(2E)-3-[6-(Acetylamino)pyridin-3-yl]-N-{2-[(2,4-dichloro-3-{[(2-methylquinolin-8-yl)oxy]methyl}phenyl)(methyl)amino]-2-oxoethyl}prop-2-enamide;
(2E)-3-[6-(Acetylamino)pyridin-3-yl]-N-{2-[(2,4-dichloro-3-{[(2-methylquinolin-8-yl)oxy]methyl}phenyl)amino]-2-oxoethyl}prop-2-enamide;
(2E)-N-{2-[(4-Chloro-2-cyano-3-{[(2 methylquinolin-8-yl)oxy]methyl}phenyl)(methyl)amino]-2-oxoethyl}-3-[4-(trifluoromethyl)phenyl]prop-2-enamide;
N-[4-chloro-2-cyano-3-[(2-methylquinolin-8-yl)oxymethyl]phenyl]-2-(ethylcarbamoylamino)-N-methylacetamide;
2-(4-aminobutylcarbamoylamino)-N-[4-chloro-2-cyano-3-[(2-methylquinolin-8-yl)oxymethyl]phenyl]N-methylacetamide;
4-[[2-[4-chloro-2-cyano-N-methyl-3-[(2-methylquinolin-8-yl)oxymethyl]anilino]-2-oxoethyl]carbamoylamino]butanoic acid;
(E)-N-[2-[4-chloro-2-cyano-N-methyl-3-[(2-methylquinolin-8-yl)oxymethyl]anilino]-2-oxoethyl]-3-(3-methoxyphenyl)prop-2-enamide;
(E)-N-[2-[4-chloro-2-cyano-N-methyl-3-[(2-methylquinolin-8-yl)oxymethyl]anilino]-2-oxoethyl]-3-[4-(trifluoromethyl)phenyl]prop-2-enamide;
N-[2,4-dichlor-3-[(2-methylquinolin-8-yl)oxymethyl]phenyl]-2-[5-(2,2-dimethylpropanoyl)-1-methylpyrrol-2-yl]-N-methylacetamide;
4-[(E)-3-[[(Z)-3-[2,4-dichloro-3-[(2-methylquinolin-8-yl)oxymethyl]phenyl]prop-2-enyl]amino]-3-oxoprop-1-enyl]-N-methylbenzamide;
(E)-N-[2-[2,4-dichloro-N-methyl-3-[(2-methylquinolin-8-yl)oxymethyl]anilino]-2-oxoethyl]-3-phenylprop-2-enamide hydrochloride;
2-(5-benzoyl-1-methylpyrrol-2-yl)-N-[2,4-dichloro-3-[(2-methylquinolin-8-yl)oxymethyl]phenyl]N-methylacetamide;
(E)-3-(6-acetamidopyridin-3-yl)-N-[2-[2,4-dichloro-3-[(2-methylquinolin-8-yl)oxymethyl]anilino]-2-oxoethyl]prop-2-enamide;
N-[2,4-dichloro-3-[(2-methylquinolin-8-yl)oxymethyl]phenyl]-N-methyl-2-[1-methyl-5-(thiophene-2-carbonyl)pyrrol-2-yl]acetamide;
2-[5-(cyclohexanecarbonyl)-1-methylpyrrol-2-yl]-N-[2,4-dichloro-3-[(2-methylquinolin-8-yl)oxymethyl]phenyl]-N-methylacetamide;
2-[5-(4-cyanobenzoyl)-1-methylpyrrol-2-yl]-N-[2,4-dichloro-3-[(2-methylquinolin-8-yl)oxymethyl]phenyl]acetamide;
2-[5-(4-cyanobenzoyl)-1H-pyrrol-2-yl]-N-[2,4-dichloro-3-[(2-methylquinolin-8-yl)oxymethyl]phenyl]-N-methylacetamide;
N-[2,4-dichloro-3-[(2-methylquinolin-8-yl)oxymethyl]phenyl]-N-methyl-2-[-methyl-5-(2-phenylacetyl)pyrrol-2-yl]acetamide;
2-[5-(4-aminobenzoyl)-1-methylpyrrol-2-yl]-N-[2,4-dichloro-3-[(2-methylquinolin-4-yl)oxymethyl]phenyl]-N-methylacetamide;
N-[2,4-dichloro-3-[(2-methylquinolin-8-yl)oxymethyl]phenyl]-N-methyl-3-[1-methyl-5-(pyridine-3-carbonyl)pyrrol-2-yl]propanamide;
4-[(E)-3-[[2-[2,4-dichloro-N-methyl-3-[(2-methylquinolin-8-yl)oxymethyl]anilino]-2-oxoethyl]amino]-3-oxoprop-1-enyl]-N-methylbenzamide;
(E)-3-(6-acetamidopyridin-3-yl)-N-[2-[2,4-dichloro-N-methyl-3-[(2-methylquinolin-8-yl)oxymethyl]anilino]-2-oxoethyl]prop-2-enamide;
N-[2,4-dichloro-3-[(2-methylquinolin-8-yl)oxymethyl]phenyl]-N-methyl-3-[1-methyl-5-(thiophene-2-carbonyl)pyrrol-2-yl]propanamide;
2-[5-(4-cyanobenzoyl)-1-methylpyrrol-2-yl]-N-[2,4-dichloro-3-[(2-methylquinolin-8-yl)oxymethyl]phenyl]-N-methylacetamide;
2-[5-(6-cyanopyridine-3-carbonyl)-1-methylpyrrol-2-yl]-N-[2,4-dichloro-3-[(2-methylquinolin-8-yl)oxymethyl]phenyl]-N-methylacetamide;

or a pharmaceutically acceptable salt, stereoisomer, hydrate, or solvate thereof, thereby reducing plasma extravasation in the subject.

Certain embodiments are drawn to oral formulations comprising a therapeutically effective amount of a compound having formula (I) or (II) or a pharmaceutically acceptable salt, stereoisomer, hydrate, or solvate thereof and a pharmaceutically acceptable carrier, wherein the therapeutically effective amount is between about 0.001 wt % and about 60 wt % of the oral formulation.

Certain embodiments are drawn to the use of a composition comprising a compound having formula (I) or (II) or a pharmaceutically acceptable salt, stereoisomer, hydrate, or solvate thereof for the manufacture of a medicament for the treatment and/or prevention of a B₂-bradykinin receptor mediated angioedema.

### BRIEF DESCRIPTION OF THE FIGURES

FIG. 1 depicts a synthesis scheme for 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711).

FIG. 2 is a graph showing Evans blue concentration (μg/mg)±SEM (standard error of the mean) of tissue in C57BL/6J (wild-type) mice following intravenous administration of 1-((4-chloro-3-(((4-(4-fluoro-1-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711). *Significant differences to vehicle (p<0.05, Kruskal-Wallis One Way ANOVA with multiple comparisons versus control group (Dunn's Method), n=8) Insert shows the value distribution of single animals.

FIG. 3 is a graph showing Evans blue concentration (μg/mg)±SEM (standard error of the mean) of tissue in C57BL/6J (wild-type) mice following oral administration of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711). * *Significant differences to vehicle p<0.05, (Mann-Whitney Rank Sum Test) JSM11938=HGT3711=1-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile

FIG. 4 is a graph showing Evans blue concentration (mg/mL)±SEM of bladder extract in C1-INH mice following oral administration of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711).

FIG. 5 is a graph showing Evans blue concentration (mg/mL)±SEM of bladder extract in C1-INH KO (knockout) mice following oral administration of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711).

FIG. 6 is a graph showing the results of ex vive efficacy of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (JSM1938/HGT3711) in a human umbilical vein assay.

FIG. 7 is a graph showing average plasma concentrations versus time following orally administered 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (HGT3711) in female CD-1 mice.

FIG. 8 shows average plasma concentration versus time following orally administered 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (HGT3711) in male and female Wistar and Sprague-Dawley rats (Formulation 1—top; Formulation 2—bottom).

FIG. 9 is a graph showing individual plasma concentration (ng/mL) of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (HGT3711) versus time following oral administration in female Yucatan mini-pigs at 10 mg/kg (Should this be swapped out with a later graph)?

FIG. 10 shows representative chromatograms of HGT3711 incubated for 4 hours with mouse, rat, mini-pig and human hepatocytes. * HPLC retention time of HGT3711 (JSM11938) was 35 minutes.

FIG. 11 depicts the structures of metabolites of 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile.

### DETAILED DESCRIPTION

Certain embodiments are drawn to methods of treating a B₂-bradykinin receptor mediated angioedema (such as, hereditary angioedema) by administering a therapeutically effective amount of a composition containing a 8-(heteroarylmethoxy)quinoline or 8-(arylmethoxy)quinoline or a pharmaceutically acceptable salt, a stereoisomer, a hydrate, or a solvate thereof. These compounds can act as selective modulators (e.g., antagonists) of B₂-bradykinin receptors and can result in reduced plasma extravasation in a subject after they are administered.

B₂-bradykinin receptor modulators (e.g., antagonists) provided herein can exhibit high activity on human B₂-bradykinin receptor (i.e., an inhibition constant (IC₅₀) for competition with binding of labeled bradykinin (BK) to human B₂-bradykinin receptor of less than about 5 micromolar) or very high activity on human B₂-bradykinin receptor (i.e., an IC₅₀ for competition with the binding of labeled BK to human B₂-bradykinin receptor of less than about 50 nanomolar). In certain embodiments, such modulators exhibit a high activity on B₂-bradykinin receptors of species other than human, e.g., rat, mouse, gerbil, guinea pig, rabbit, dog, cat, pig, or cynomolgus monkey.

The activity of the B₂-bradykinin receptor modulators can be assessed using appropriate in vitro assays. For instance, the IC₅₀ values of the modulators for B₂-bradykinin receptor can be determined via a radioligand binding assay. Inhibitory effects of the B₂-bradykinin receptor modulators provided herein for B₂-bradykinin receptor can be determined, for example, via a calcium mobilization assay. B₂-bradykinin receptor modulators can have an IC₅₀ (half-maximal inhibitory concentration) of about 5 micromolar or less, about 500 nM or less, about 50 nM or less, about 10 nM or less, or about 1 nanomolar or less in the assays mentioned above. In embodiments, a compound having formula (I) or (II) can have a half maximal inhibitory concentration (IC₅₀) for competition with the binding of labeled bradykinin to human B₂-bradykinin receptor of less than about 50 nanomolar, less than about 10 nanomolar, or less than about 5 nanomolar to B₂-bradykinin receptor.

Certain embodiments comprise administering pharmaceutical compositions comprising at least one B₂-bradykinin receptor modulator as described herein, in combination with a physiologically acceptable carrier or excipient. Processes for preparing such pharmaceutical compositions are also provided. Such compositions can be useful in the treatment of B₂-bradykinin receptor mediated angioedema (e.g., HAE).

Compounds are generally described herein using standard nomenclature. For compounds having asymmetric centers, it should be understood that (unless otherwise specified) all of the optical isomers and mixtures thereof are encompassed. Compounds with two or more asymmetric elements can also be present as mixtures of diastereomers. In addition, compounds with carbon-carbon double bonds can occur in Z- and E-forms, with all isomeric forms of the compounds being included in embodiments unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹C, ¹³C, and ¹⁴C.

Compounds according to the formulas provided herein, which have one or more stereogenic centers, have an enantiomeric excess of at least 50%. For example, such compounds can have an enantiomeric excess of at least 60%, 70%, 80%, 85%, 900%, 95%, or 98%. Some embodiments of the compounds have an enantiomeric excess of at least 99%. It will be apparent that single enantiomers (optically active forms) can be obtained by asymmetric synthesis, synthesis from optically pure precursors or by resolution of the racemates. Resolution of the racemates can be accomplished, for example, by conventional methods such as crystallization in the presence of a resolving agent, or chromatography, using, for example a chiral HPLC column.

Certain compounds are described herein using a general formula that includes variables (e.g., R₁-R₉). Unless otherwise specified, each variable within such a formula is defined independently of any other variable, and any variable that occurs more than one time in a formula is defined independently at each occurrence. Thus, for example, if a group is shown to be substituted with 0-2 R*, the group can be unsubstituted or substituted with up to two R* groups and R* at each occurrence is selected independently from the definition of R*. Also, combinations of substituents and/or variables are permissible only if such combinations result in stable compounds (i.e., compounds that can be isolated, characterized and tested for biological activity).

The terms “8-(arylmethoxy)quinoline” and “8-(heteroarylmethoxy)quinoline”, as used herein, refer to compounds of formula (I) or (II) provided herein (described below), as well as pharmaceutically acceptable salts, stereoisomers, hydrates, and solvates thereof. It will be apparent that such compounds can be further substituted as indicated.

Formula (I) is as follows:

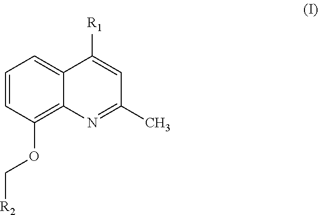

R₁ can be hydrogen; an optionally substituted alkyl; optionally substituted alkenyl; 5-membered heterocycloalkyl having from 1 to 3 heteroatoms each independently selected from N, O or S, or cycloalkyl, wherein said 5-membered heterocycloalkyl or cycloalkyl can be substituted with from 0 to 3 substituents each independently selected from halogen atom, oxygen atom, hydroxy, cyano, amino, nitro, mercapto, alkyl, alkenyl, alkynyl, heteroalkyl, cycloalkyl, heterocycloalkyl, alkylcycloalkyl, heteroalkylcycloalkyl, aryl, heteroaryl, aralkyl, or heteroaralkyl; or a 5-membered heteroaryl having from 1 to 4 heteroatoms each independently selected from N, O or S, wherein said 5-membered heteroaryl is substituted with from 0 to 3 substituents each independently selected from halogen atom, oxygen atom, hydroxy, cyano, amino, nitro, mercapto, alkyl, alkenyl, alkynyl, heteroalkyl, optionally substituted aryl or optionally substituted heteroaryl.

In certain embodiments, R₁, can be H, a C₁-C₆ alkyl (e.g., a methyl group, an ethyl group, a propy group, a butyl group, a pentyl group, or a hexyl group)

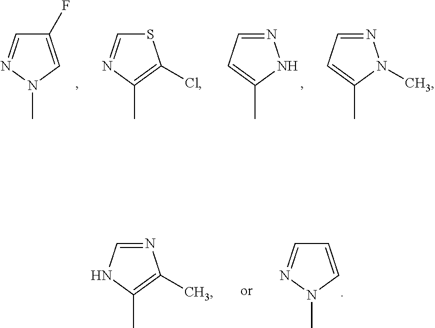

In some embodiments, R₁ can be H or

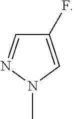

In embodiments R₂ can be a 6-membered aryl or 6-membered heteroaryl, wherein the 6-membered heteroaryl comprises 1 nitrogen atom. The 6-membered aryl or heteroaryl can be substituted with 1 to 3 substituents each independently selected from halogen atom, oxygen atom, hydroxy, cyano, amino, nitro, mercapto, alkyl, alkenyl, alkynyl, heteroalkyl, cycloalkyl, heterocycloalkyl, alkylcycloalkyl, heteroalkylcycloalkyl, aryl, heteroaryl, aralkyl, and heteroaralkyl.

In certain embodiments R₂ can be

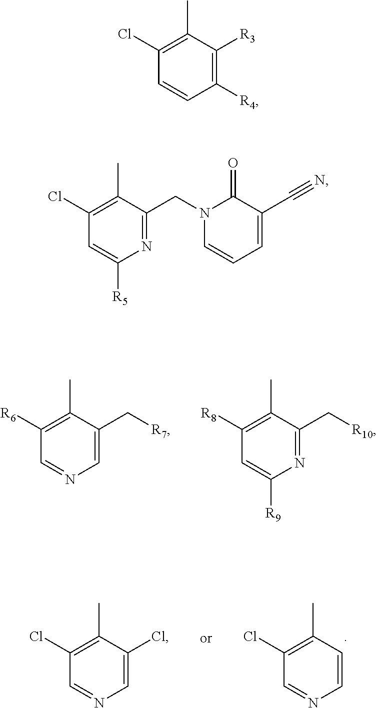

R₃, R₄, R₅, R₇, R₈, R₉ and R₁₀ can each be independently selected from a halogen atom, an oxygen atom, hydroxy, cyano, amino, nitro, mercapto, alkyl, alkenyl, alkynyl, heteroalkyl, cycloalkyl, heterocycloalkyl, alkylcycloalkyl, heteroalkylcycloalkyl, aryl, heteroaryl, aralkyl, and heteroaralkyl, and R₃ can also be selected from H in some embodiments.

In some embodiments, R₃ can be a halogen atom (such as, Cl), CN or H. In certain embodiments, R₃ can be Cl or CN. R₅ can be a methyl group, an ethyl group, a propyl group, a butyl group, a pentyl group, or a hexyl group, in certain embodiments. In certain embodiments, R₅ is a methyl group. R₆ can be a halogen atom (such as, Cl) or a C₁-C₆ alkyl (such as, CH₃), in certain embodiments.

R₄ can, in certain embodiments, have the formula

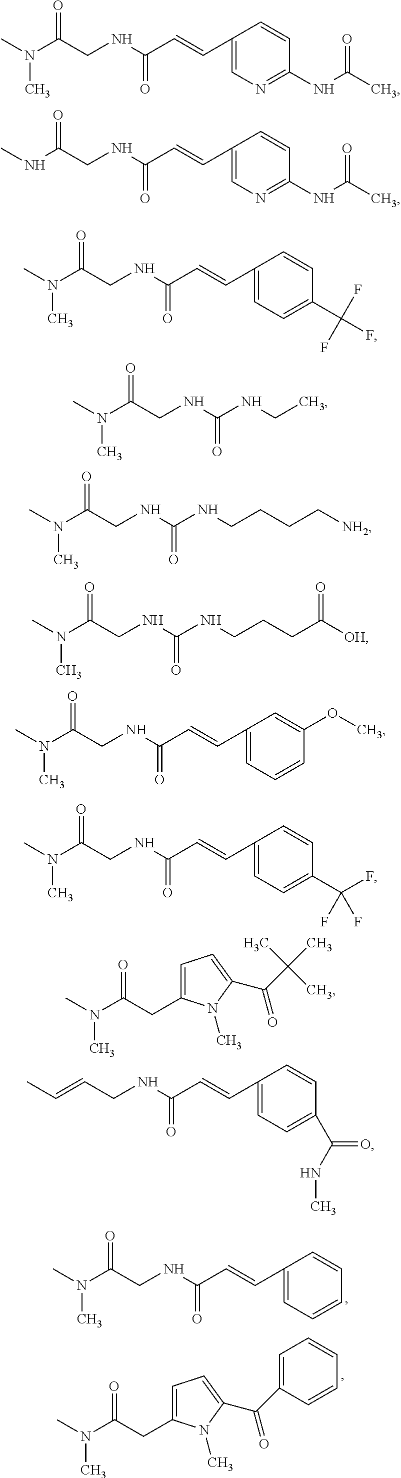

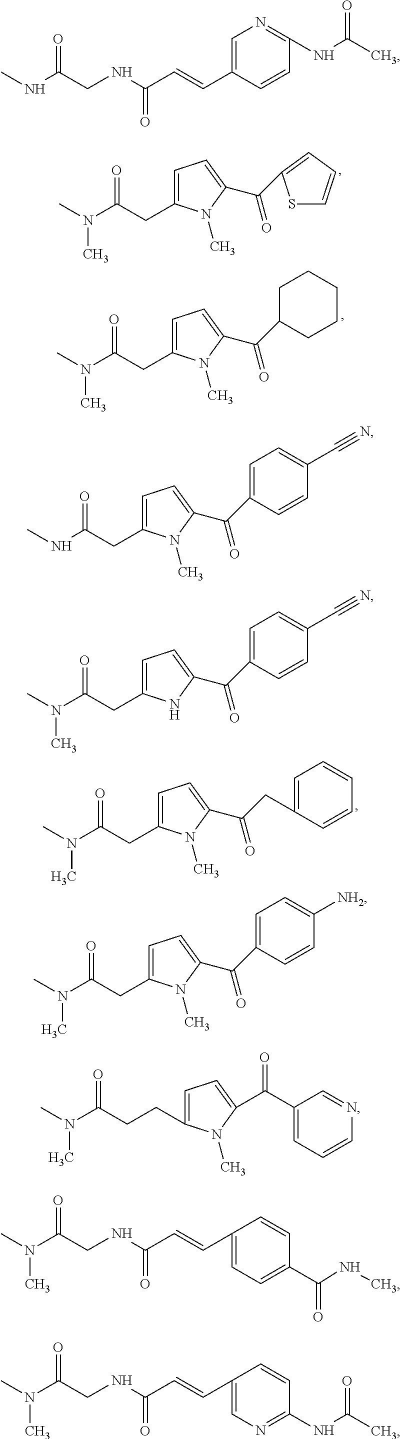

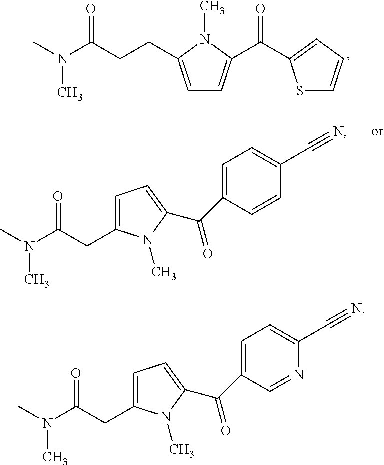

In some embodiments, R₇ can have a formula

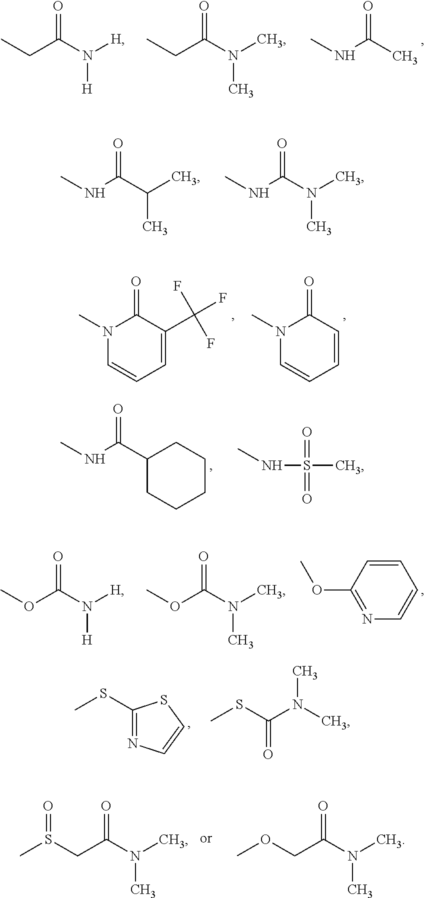

In certain embodiments, R₈ can be a halogen or a C₁-C₆ alkyl. R₅ can be Cl or CH₃ in embodiments. In some embodiments, R₉ can be H or a C₁-C₆ alkyl. R₉ can be CH₃, in certain embodiments.

R₁₀ can be selected from

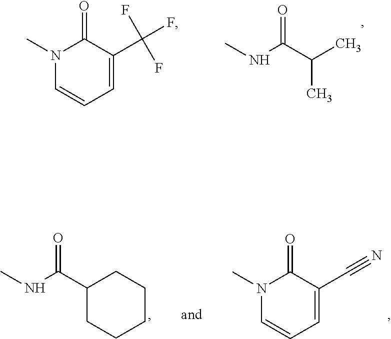

in some embodiments.

Certain embodiments are drawn to methods of treating a B₂-bradykinin receptor mediated angioedema in a subject comprising:

administering to the subject in need thereof a therapeutically effective amount of a composition comprising a compound having formula (I) or (II) or a pharmaceutically acceptable salt, stereoisomer, hydrate, or solvate thereof.

In certain embodiments the compound having formula (I) can be as follows:

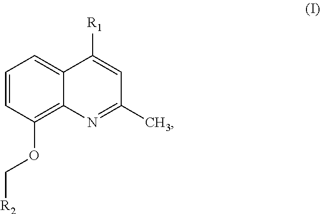

wherein R₁ is

### H or

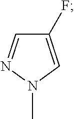

wherein R₂ is

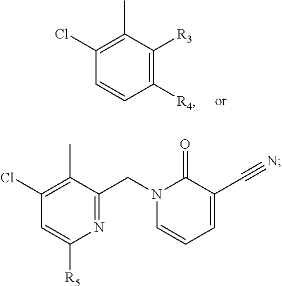

wherein R₃ is Cl or CN;
wherein R₄ is

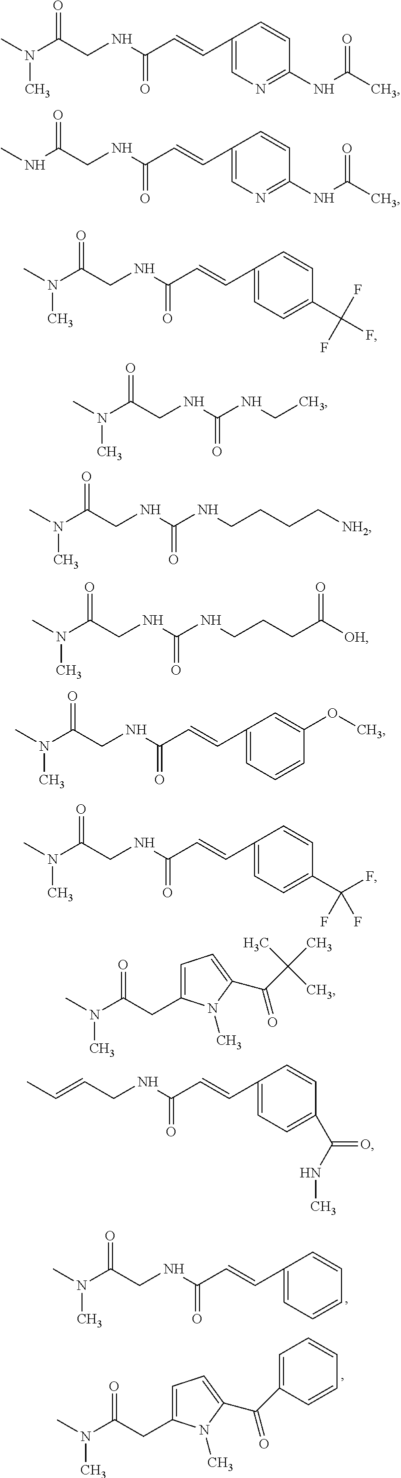

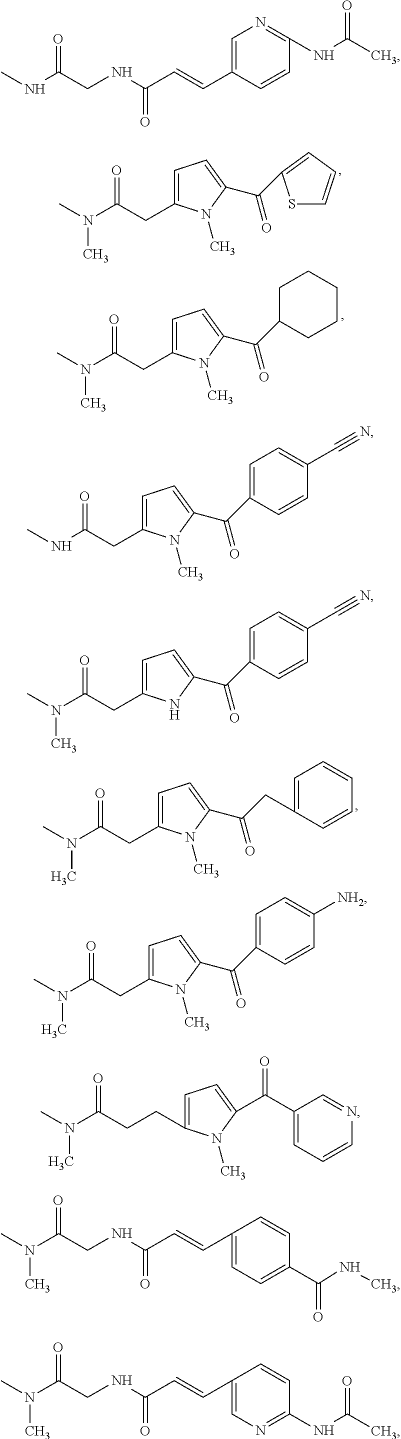

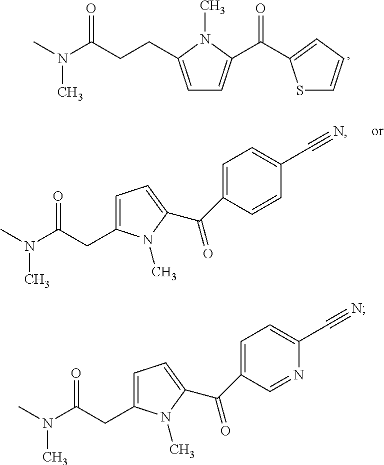

and

wherein R₅ is selected from the group consisting of H, a methyl group, an ethyl group, a propyl group, a butyl group, a pentyl group, or a hexyl group

Plasma extravasation in the subject can be reduced upon administration of the compound of formula (I) or (II) or the pharmaceutically acceptable salt, stereoisomer, hydrate, or solvate thereof. The B₂-bradykinin receptor mediated angioedema treated by embodiments can be hereditary angioedema. Certain treatment methods of embodiments can further comprise administering icatibant, ecallantide, fresh frozen plasma, C1-inhibitor, or kallikrein inhibitor to the subject, in addition to a therapeutically effective amount of a composition comprising a compound having formula (I) or (II) or a pharmaceutically acceptable salt, stereoisomer, hydrate, or solvate thereof.

Some specific examples of compounds that can be used in embodiments encompassed by formula (I) or (II) include:
11-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile;
(2E)-3-[6-(Acetylamino)pyridin-3-yl]-N-{2-[(2,4-dichloro-3-{[(2-methylquinolin-8-yl)oxy]methyl}phenyl)(methyl)amino]-2-oxoethyl}prop-2-enamide;
(2E)-3-[6-(Acetylamino)pyridin-3-yl]-N-{2-[(2,4-dichloro-3-{[(2-methylquinolin-8-yl)oxy]methyl}phenyl)amino]-2-oxoethyl}prop-2-enamide;
(2E)-N-{2-[(4-Chlor-2-cyano-3-{[(2-methylquinolin-8-yl)oxy]methyl}phenyl)(methyl)amino]-2-oxoethyl}-3-[4-(trifluoromethyl)phenyl]prop-2-enamide;

N-[4-chloro-2-cyano-3-[(2-methylquinolin-8-yl)oxymethyl]phenyl]-2-(ethylcarbamoylamino)-N-methylacetamide;
2-(4-aminobutylcarbamoylamino)-N-[4-chloro-2-cyano-3-[(2-methylquinolin-8-yl)oxymethyl]phenyl]-N-methylacetamide;
4-[[2-[4-chloro-2-cyano-N-methyl-3-[(2-methylquinolin-4-yl)oxymethyl]anilino]-2-oxoethyl]carbamoylamino]butanoic acid;
(E)-N-[2-[4-chloro-2-cyano-N-methy-3-[(2-methylquinolin-8-yl)oxymethyl]anilino]-2-oxoethyl]-3-(3-methoxyphenyl)prop-2-enamide;
(E)-N-[2-[4-chloro-2-cyano-N-methyl-3-[(2-methylquinolin-8-yl)oxymethyl]anilino]-2-oxoethyl]-3-[4-(trifluoromethyl)phenyl]prop-2-enamide;
N-[2,4-dichloro-3-[(2-methylquinolin-8 yl)oxymethyl]phenyl]-2-[5-(2,2-dimethylpropanoyl)-1-methylpyrrol-2-yl]-N-methylacetamide;
4-[(E)-3-[[(Z)-3-[2,4-dichloro-3-[(2-methylquinolin-8-yl)oxymethyl]phenyl]prop-2-enyl]amino]-3-oxoprop-1-enyl]-N-methylbenzamide;
(E)-N-[2-[2,4-dichloro-N-methyl-3-[(2-methylquinolin-8-yl)oxymethyl]anilino]-2-oxoethyl]-3-phenylprop-2-enamide hydrochloride;
2-(5-benzoyl-1-methylpyrrol-2-yl)-N-[2,4-dichloro-3-[(2-methylquinolin-8-yl)oxymethyl]phenyl]-N-methylacetamide;
(E)-3-(6-acetamidopyridin-3-yl)-N-[2-[2,4-dichloro-3-[(2-methylquinolin-8-yl)oxymethyl]anilino]-2-oxoethyl]prop-2-enamide;
N-[2,4-dichloro-3[(2-methylquinolin-8-yl)oxymethyl]phenyl]-N-methyl-2-[1-methyl-5-(thiophene-2-carbonyl)pyrrol-2-yl]acetamide;
2-[5-(cyclohexanecarbonyl)-1-methylpyrrol-2-yl]-N-[2,4-dichloro-3-[(2-methylquinolin-8-yl)oxymethyl]phenyl]-N-methylacetamide;
2-[5-(4-cyanobenzoyl)-1-methylpyrrol-2-yl]-N-[2,4-dichloro-3-[(2-methylquinolin-8-yl)oxymethyl]phenyl]acetamide;
2-[5-(4-cyanobenzoyl)-1H-pyrrol-2-yl]-N-[2,4-dichloro-3-[(2-methylquinolin-8-yl)oxymethyl]phenyl]-N-methylacetamide;
N-[2,4-dichloro-3-[(2-methylquinolin-8yl)oxymethyl]phenyl]-N-methyl-2-[1-methyl-5-(2-phenylacetyl)pyrrol-2-yl]acetamide;
2-[5-(4-aminobenzoyl)-1-methylpyrrol-2-yl]-N-[2,4-dichloro-3-[(2-methylquinolin-8-yl)oxymethyl]phenyl]-N-methylacetamide;
N-[2,4-dichloro-3-[(2-methylquinolin-8-yl)oxymethyl]phenyl]-N-methyl-3-[1-methyl-5-(pyridine-3-carbonyl)pyrrol-2-yl]propanamide;
4-[(E)-3-[[2-[2,4-dichloro-N-methyl-3-[(2-methylquinolin-8-yl)oxymethyl]anilino]-2-oxoethyl]amino]-3-oxoprop-1-enyl]-N-methylbenzamide;
(E)-3-(6-acetamidopyridin-3-yl)-N-[2-[2,4-dichloro-N-methyl-3-[(2-methylquinolin-8-yl)oxymethyl]anilino]-2-oxoethyl]prop-2-enamide;
N-[2,4-dichloro-3-[(2-methylquinolin-8-yl)oxymethyl]phenyl]N-methyl-3[-methyl-5-(thiophene-2-carbonyl)pyrrol-2-yl]propanamide;
2-[5-(4-cyanobenzoyl)-1-methylpyrrol-2-yl]-N-[2,4-dichloro-3-[(2-methylquinolin-8-yl)oxymethyl]phenyl]-N-methylacetamide;
2-[5-(6-cyanopyridine-3-carbonyl)-1-methylpyrrol-2-yl]-N-[2,4-dichloro-3-[(2-methylquinolin-8-yl)oxymethyl]phenyl]-N-methylacetamide;

or pharmaceutically acceptable salts, stereoisomers, hydrates, or solvates thereof.

One example of a compound encompassed by formula (I) has formula II

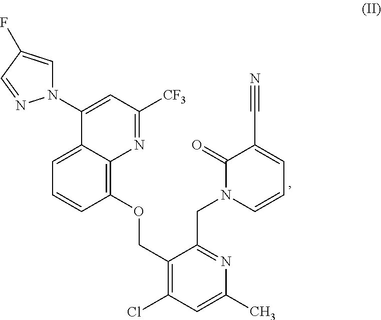 [SMILES: Cc1cc(Cl)c(COc2cccc3c(-n4cc(F)cn4)cc(C(F)(F)F)nc23)c(Cn2cccc(C#N)c2=O)n1]

1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile. Certain embodiments are drawn to methods of treating a B₂-bradykinin receptor mediated angioedema in a subject comprising administering to the subject in need thereof a therapeutically effective amount a composition comprising a compound having formula (II) or a pharmaceutically acceptable salt, stereoisomer, hydrate, or solvate thereof, thereby reducing plasma extravasation in the subject.

Certain embodiments include compositions comprising 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (HGT3711) or a pharmaceutically acceptable salts, stereoisomers, hydrates, or solvates thereof. 1-1-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (HGT3711) can be orally bioavailable and act as a B₂-bradykinin receptor antagonist in the treatment of B₂-bradykinin receptor mediated angioedema, in embodiments.

In embodiments, the 8-(heteroarylmethoxy)quinoline or 8-(arylmethoxy)quinoline or a pharmaceutically acceptable salt, a stereoisomer, a hydrate, or a solvate thereof (e.g., a compound having formula (I) or (II)) can be a small molecule. A small molecule is a low molecular weight (<800 Daltons) organic compound that may serve as an enzyme substrate or regulator of biological processes (e.g., B₂-bradykinin receptor antagonist). The upper molecular weight limit for a small molecule is about 800 Daltons which allows for the possibility to rapidly diffuse across cell membranes so that they can reach intracellular sites of action. In addition, this molecular weight cutoff is a necessary but insufficient condition for oral bioavailability. Biopolymers such as nucleic acids, proteins, and polysaccharides (such as starch or cellulose) are not small molecules. Compounds having formula (I) or (II) can have a molecular weight less than about 650 Daltons, less than about 600 Daltons, or less than about 525 Daltons in embodiments.

Certain embodiments are drawn to the therapeutic use of (a) compounds of formula (I) or (II), their pharmaceutically acceptable salts, stereoisomers, solvates or hydrates and also (b) formulations and pharmaceutical compositions containing the same. Some embodiments also relate to the use of compositions comprising a compound having formula (I) or (I), a pharmaceutically acceptable salt, stereoisomer, solvate or hydrate thereof as an active ingredient in the preparation or manufacture of a medicament for the treatment and/or prevention of a B₂-bradykinin receptor mediated angioedema.

A “pharmaceutically acceptable salt” of a compound disclosed herein is an acid or base salt that is generally considered in the art to be suitable for use in contact with the tissues of human beings or animals without excessive toxicity or carcinogenicity, and without irritation, allergic response, or other problem or complication, in some embodiments. Such salts include mineral and organic acid salts of basic residues such as amines, as well as alkali or organic salts of acidic residues such as carboxylic acids.

Suitable pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to −4 (i.e., 0, 1, 2, 3, or 4) and the like. Similarly, pharmaceutically acceptable cations include, but are not limited to sodium, potassium, calcium, aluminum, lithium, and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Nonaqueous media, such as ether, ethyl acetate, ethanol, isopropanol or acetonitrile, can be used for preparation of a salt in some embodiments.

It will be apparent that each compound of formula I can, but need not, be present as a hydrate, solvate or non-covalent complex. In addition, the various crystal forms and polymorphs are within the scope of embodiments described herein, as are prodrugs of the compounds of formula (I) or (II) provided herein.

A “prodrug” is a compound that differs structurally from 8-(heteroarylmethoxy)quinoline and 8-(arylmethoxy)quinoline compounds provided herein and that is modified in vivo, following administration to a subject or patient, to produce a compound of formula I provided herein. For example, a prodrug can be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein can be prepared by modifying functional groups present in the compounds in such a way that the modifications are cleaved in vivo to generate the parent compounds.
A “substituent,” as used herein, refers to a molecular moiety that is covalently bonded to an atom within a molecule of interest. For example, a “ring substituent” can be a moiety such as a halogen, alkyl group, haloalkyl group, hydroxy, cyano, amino, nitro, mercapto, or other substituent described herein that is covalently bonded to an atom that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., ═O), then 2 hydrogens on the atom are replaced. An oxo group that is a substituent of an aromatic carbon atom results in a conversion of —CH— to —C(═O)— and a loss of aromaticity. For example a pyridyl group substituted by oxo is a pyridone.

The expression “alkyl” refers to a saturated, straight-chain or branched hydrocarbon group that contains from 1 to 20 carbon atoms, from 1 to 12 carbon atoms, or from 1 to 6 carbon atoms, for example a methyl, ethyl, propyl, iso-propyl, n-butyl, iso-butyl, sec-butyl, tert-butyl, n-pentyl, n-hexyl, 2,2-dimethylbutyl or n-octyl group.

The expressions “alkenyl” and “alkynyl” refer to at least partially unsaturated, straight-chain or branched hydrocarbon groups that contain from 2 to 20 carbon atoms, from 2 to 12 carbon atoms, or from 2 to 6 carbon atoms, for example an ethenyl, allyl, acetylenyl, propargyl, isoprenyl or hex-2-enyl group. Alkenyl groups can have one or two double bonds and alkynyl groups have one or two triple bonds, in certain embodiments.

Furthermore, the terms “alkyl”, “alkenyl” and “alkynyl” refer to groups in which one or more hydrogen atoms have been replaced each independently of the others by a halogen atom (such as, F or Cl) such as, for example, a 2,2,2-trichloroethyl or a trifluoromethyl group.

The expression “heteroalkyl” refers to an alkyl, alkenyl or alkynyl group (for example heteroalkenyl, heteroalkynyl) in which one or more carbon atoms have been replaced each independently of the others by an oxygen, nitrogen, phosphorus, boron, selenium, silicon or sulphur atom. The expression heteroalkyl furthermore refers to a carboxylic acid or to a group derived from a carboxylic acid such as, for example, acyl, acylalkyl, alkoxycarbonyl, acyloxy, acyloxyalkyl, carboxyalkylamide, alkylcarbamoylalkyl, alkylcarbamoyloxyalkyl, alkylureidoalkyl, or alkoxycarbonyloxy.

Examples of “heteroalkyl” groups are groups of formulae —S—Y^{a}-L, —S—Y^{a}—CO—NR^{a}R^{b}, —Y^{a}—NR^{c}—CO—NR^{a}R^{b}, —Y^{a}—NR^{c}—CO—O—R^{c}, —Y^{a}—NR^{c}—CO—R^{c}, —Y^{a}—O—CO—NR^{a}R^{b}, —Y^{a}—CO—NR^{a}R^{b}, —O—Y^{a}—CO—NR^{a}R^{b}, —Y^{a}—NR^{c}—CO-L, —Y^{a}-L, —Y^{a}—O—CO—O—R^{c}, —Y^{a}—O—CO—R^{c}, R^{c}—O—Y^{a}—, R^{c}—S—Y^{a}, R^{a}—N(R^{b})—Y^{a}—, R^{c}—CO—Y^{a}—, R^{c}—O—CO—Y^{a}—, R^{c}—CO—O—Y^{a}—, R^{c}—CO—N(R^{b})—Y^{a}—, R^{a}—N(R^{b})—CO—Y^{a}—, R^{c}—SO—Y^{a}—, R^{c}SO₂—Y^{a}—, —Y^{a}—NR^{c}—SO₂—NR^{a}R^{b}, —Y^{a}—SO₂—NR^{a}R^{b}, —Y^{a}—NR^{c}—SO₂—R^{c}, R^{a}—O—CO—N(R^{b})—Y^{a}—, R^{a}—N(R^{b})—C(═NR^{d})—N(R^{c})—Y^{a}—, R^{c}—S—CO—Y^{a}—, R—CO—S—Y^{a}, R^{c}—S—CO—N(R^{b})—Y^{a}—, R^{a}—N(R^{b})—CO—S—Y^{a}—, R^{c}—S—CO—O—Y^{a}, R^{c}—O—CO—S—Y^{a}—, R^{c}—S—CO—S—Y^{a}—; R^{a} being a hydrogen atom, a C₃-C₆ alkyl, a C₂-C₆ alkenyl, a C₂-C₆ alkynyl, or is joined to R^{b} to form a 4- to 10-membered cycloalkyl or heterocycloalkyl; R^{b} being a hydrogen atom, a C₁-C₆ alkyl, a C₂-C₆ alkenyl or a C₂-C₆ alkynyl, or taken together with R^{a} to form a 4- to 10-membered cycloalkyl or heterocycloalkyl; R^{c} being a hydrogen atom, an optionally substituted C₁-C₆ alkyl, an optionally substituted C₂-C₆ alkenyl or an optionally substituted C₂-C₆ alkynyl; Rd being a hydrogen atom, a C₁-C₆ alkyl, a C₂-C₆ alkenyl or a C₂-C₆ alkynyl; L being a cycloalkyl, heterocycloalkyl, alkylcycloalkyl, heteroalkylcycloalkyl, aryl, optionally substituted heteroaryl, aralkyl, or heteroaralkyl; and Y^{a} being a bond, a C₁-C₆ alkylene, a C₂-C₆ alkenylene or a C₂-C₆ alkynylene group; each heteroalkyl group containing at least one carbon atom and it being possible for one or more hydrogen atoms to have been replaced by fluorine or chlorine atoms. Specific examples of heteroalkyl groups are methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, iso-propylethylamino, methylaminomethyl, ethylaminomethyl, diisopropylaminoethyl, enol ether, dimethylaminomethyl, dimethylaminoethyl, acetyl, propionyl, butyryloxy, acetyloxy, methoxycarbonyl, ethoxycarbonyl, isobutyrylamino-methyl, N-ethyl-N-methylcarbamoyl and N-methylcarbamoyl. Further examples of heteroalkyl groups are nitrile, isonitrile, cyanate, thiocyanate, isocyanate, isothiocyanate and alkylnitrile groups. An example of a hetero-alkylene group is a group of formula —CH₂CH(OH)— or —CONH—.

The expression “cycloalkyl” refers to a saturated or partially unsaturated cyclic group that contains one or more rings, containing from 3 to 14 ring carbon atoms, from 3 to 10 ring carbon atoms, or 3 to 6 ring carbon atoms. In an embodiment a partially unsaturated cyclic group has one, two or more double bonds, such as a cycloalkenyl group. The expression cycloalkyl refers furthermore to groups in which one or more hydrogen atoms have been replaced each independently of the others by fluorine, chlorine, bromine or iodine atoms or by OH, ═O, SH, ═S, NH₂, ═NH, CN or NO₂ groups, thus, for example, cyclic ketones such as, for example, cyclohexanone, 2-cyclohexenone or cyclopentanone. Further specific examples of a cycloalkyl group are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetralin, cyclopentylcyclohexyl, fluorocyclohexyl or cyclohex-2-enyl group.

The expression “heterocycloalkyl” refers to a cycloalkyl group as defined above in which one or more ring carbon atoms have been replaced each independently of the others by an oxygen, nitrogen, silicon, selenium, phosphorus or sulphur atom. A heterocycloalkyl group has 1 or 2 rings containing from 3 to 10 ring atoms. The expression heterocycloalkyl refers furthermore to groups in which one or more hydrogen atoms have been replaced each independently of the others by fluorine, chlorine, bromine or iodine atoms or by OH, ═O, SH, ═S, NH₂, ═NH, CN or NO₂ groups. Examples are a piperidyl, piperazinyl, morpholinyl, urotropinyl, pyrrolidinyl, tetrahydrothiophenyl, tetrahydropyranyl, tetrahydrofuryl or 2-pyrazolinyl group and also a lactam, a lactone, a cyclic imide and a cyclic anhydride.

The expression “alkylcycloalkyl” refers to a group containing both cycloalkyl and also an alkyl, alkenyl or alkynyl group in accordance with the above definitions, for example alkyl-cycloalkyl, cycloalkylalkyl, alkylcycloalkenyl, alkenylcycloalkyl and alkynylcycloalkyl groups. An alkylcycloalkyl group can contain a cycloalkyl group that contains one or two ring systems having from 3 to 10 carbon atoms, and one or two alkyl, alkenyl or alkynyl groups having 1 or 2 to 6 carbon atoms, the cyclic groups being optionally substituted.

The expression “heteroalkylcycloalkyl” refers to alkylcycloalkyl groups as defined above in which one or more carbon atoms have been replaced each independently of the others by an oxygen, nitrogen, silicon, selenium, phosphorus or sulphur atom. A heteroalkylcycloalkyl group can contain 1 or 2 ring systems having from 3 to 10 ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being optionally substituted and saturated or mono-, di- or tri-unsaturated.

The expression “aryl” refers to an aromatic group that contains one or more rings containing from 6 to 14 ring carbon atoms, or from 6 to 10 ring carbon atoms. The expression aryl refers furthermore to groups in which one or more hydrogen atoms have been replaced each independently of the others by fluorine, chlorine, bromine or iodine atoms or by OH, SH, NH₂, CN or NO₂ groups. Examples are a phenyl, naphthyl, biphenyl, 2-fluorophenyl, anilinyl, 3-nitrophenyl or 4-hydroxyphenyl group.

The expression “heteroaryl” refers to an aromatic group that contains one or more rings containing from 5 to 14 ring atoms, or from 5 to 10 ring atoms, and contains one or more oxygen, nitrogen, phosphorus or sulphur ring atoms. The expression heteroaryl refers furthermore to groups in which one or more hydrogen atoms have been replaced each independently of the others by fluorine, chlorine, bromine or iodine atoms or by OH, ═O, SH, NH₂, ═NH, CN or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl.

The expression “aralkyl” refers to a group containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetralin, dihydronaphthalene, indanone, phenylcyclopentyl, cumene, cyclohexylphenyl, fluorene and indan. An aralkyl group contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression “heteroaralkyl” refers to an aralkyl group as defined above in which one or more carbon atoms have been replaced each independently of the others by an oxygen, nitrogen, silicon, selenium, phosphorus, boron or sulphur atom, that is to say to groups containing both aryl or heteroaryl and also alkyl, alkenyl, alkynyl and/or heteroalkyl and/or cycloalkyl and/or heterocycloalkyl groups in accordance with the above definitions. A heteroaralkyl group can contain one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced each independently of the others by oxygen, sulphur or nitrogen atoms.

Examples of heteroaralkyl groups are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl, heteroarylheteroalkylcycloalkyl, heteroarylheteroalkylcycloalkenyl, heteroalkylheteroarylalkyl and heteroarylheteroalkylheterocycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, and 2-, 3- or 4-carboxyphenylalkyl groups.

The expressions cycloalkyl, heterocycloalkyl, alkylcycloalkyl, heteroalkylcycloalkyl, aryl, heteroaryl, aralkyl and heteroaralkyl refer to groups in which one or more hydrogen atoms of such groups have been replaced each independently of the others by fluorine, chlorine, bromine or iodine atoms or by OH, ═O, SH, ═S, NH₂, ═NH, CN or NO₂ groups.

The expression “optionally substituted” refers to groups in which one or more hydrogen atoms have been replaced each independently of the others by hydrogen, fluorine, chlorine, bromine or iodine atoms or by OH, ═O, SH, ═S, NH₂, ═NH, CN or NO₂ groups. This expression refers furthermore to groups in which one or more hydrogen atoms have been replaced each independently of the others by unsubstituted C₁-C₆ alkyl, unsubstituted C₂-C₆ alkenyl, unsubstituted C₂-C₆ alkynyl, unsubstituted C₁-C₆ heteroalkyl, unsubstituted C₃-C₁₀ cycloalkyl, unsubstituted C₂-C₉ heterocycloalkyl, unsubstituted C₆-C₁₀ aryl, unsubstituted C₁-C₉ heteroaryl, unsubstituted C₇-C₁₂ aralkyl or unsubstituted C₂-C₁₁ heteroaralkyl groups.

As used herein a wording defining the limits of a range of length such as, e.g., “from 1 to 5” means any integer from 1 to 5, i.e., 1, 2, 3, 4 and 5. In other words, any range defined by two integers explicitly mentioned is meant to comprise and disclose any integer defining said limits and any integer comprised in said range.

Certain embodiments can comprise isotopes of atoms of the described compounds. Isotopes are atoms having the same atomic number but different mass numbers. For example, tritium and deuterium are isotopes of hydrogen. Examples for carbon isotopes are ¹¹C, ¹³C and ¹⁴C.

The therapeutic use of compounds of formula (I) or (II), their pharmaceutically acceptable salts, stereoisomers, solvates or hydrates and also formulations and pharmaceutical compositions can be used in embodiments for treating a B₂-bradykinin receptor mediated angioedema in a subject. Certain embodiments are drawn to the use of those compounds of formula (I) or (II) as active ingredients in the preparation or manufacture of a medicament.

The pharmaceutical compositions can comprise at least one compound of formula (I) or (II) and, optionally, one or more carrier substances, excipients and/or adjuvants. Pharmaceutical compositions can additionally comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. In embodiments the pharmaceutical compositions can comprise one or more of surfactants, tonicity agents (e.g., NaCl), buffers (e.g., phosphate or citrate buffer), salts, preservatives (e.g., sodium edetate), co-solvent, and viscosity building agents.

Furthermore, one or more other active ingredients can (but need not) be included in the pharmaceutical compositions provided herein. For instance, the 8-(heteroarylmethoxy)quinoline and 8-(arylmethoxy)quinolone compounds can be employed in combination with icatibant (injectable icatibant=Firazyr), ecallantide, C1-inhibitor, or kallikrein inhibitor.

Pharmaceutical compositions can be formulated for any appropriate manner of administration, including, for example, topical (e.g., transdermal or ocular), oral, buccal, nasal, vaginal, rectal or parenteral administration. The term parenteral as used herein includes subcutaneous, intradermal, intravascular (e.g., intravenous), intramuscular, spinal, intracranial, intrathecal, intraocular, periocular, intraorbital, intrasynovial and intraperitoneal injection, as well as any similar injection or infusion technique. In certain embodiments, compositions are in a form suitable for oral use. Such forms include, for example, tablets, pills, troches, lozenges, aqueous or oily suspensions, dispersible powders or granules, emulsion, hard or soft capsules, solutions, or syrups or elixirs. Within yet other embodiments, compositions provided herein can be formulated as a lyophilizate. Some embodiments include compositions in a form suitable for sublingual administration. The pharmaceutical composition can have a pH of less than about 7, less than about than about 6, less than about 5, less than about 4, less than about 3 or less than about 2 in embodiments.

Compositions intended for oral or sublingual use can further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient can be mixed with an inert solid diluent (e.g., calcium carbonate, calcium phosphate or kaolin), or as soft gelatin capsules wherein the active ingredient can be mixed with water or an oil medium (e.g., peanut oil, liquid paraffin or olive oil). In embodiments oral formulations can comprise a therapeutically effective amount of a compound having formula (I) or (II) or a pharmaceutically acceptable salt, stereoisomer, hydrate, or solvate thereof and a pharmaceutically acceptable carrier, wherein the therapeutically effective amount can be between about 0.001 wt % and about 60 wt %; about 0.01 wt % and about 55 wt %; about 0.1 wt % and about 60 wt %; about 1 wt % and about 50 wt % of the oral formulation. In some embodiment the oral formulation can further comprise hydroxyl propyl methyl cellulose acetate succinate. The oral formulation can be in the form of a spray-dried dispersion in certain embodiments.

Aqueous suspensions contain the active ingredient(s) in admixture with excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions can also comprise one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil (e.g., arachis oil, olive oil, sesame oil or coconut oil) or in a mineral oil such as liquid paraffin. The oily suspensions can contain a thickening agent such as beeswax, hard paraffin or cetyl alcohol. Sweetening agents such as those set forth above, and/or flavoring agents can be added to provide palatable oral preparations. Such suspensions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, can also be present.

Pharmaceutical compositions can also be in the form of oil-in-water emulsions. The oily phase can be a vegetable oil (e.g., olive oil or arachis oil), a mineral oil (e.g., liquid paraffin) or a mixture thereof Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monooleate) and condensation products of partial esters derived from fatty acids and hexitol with ethylene oxide (e.g., polyoxyethylene sorbitan monooleate). An emulsion can also comprise one or more sweetening and/or flavoring agents.

Syrups and elixirs can be formulated with sweetening agents, such as glycerol, propylene glycol, sorbitol or sucrose. Such formulations can also comprise one or more demulcents, preservatives, flavoring agents and/or coloring agents.

Compounds can be formulated for local or topical administration, such as for topical application to the skin or mucous membranes, such as in the eye. Formulations for topical administration can comprise a topical vehicle combined with active agent(s), with or without additional optional components. Suitable topical vehicles and additional components are well known in the art, and it will be apparent that the choice of a vehicle can be adjusted in view of the particular physical form and mode of delivery. Topical vehicles include water, organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices.

A composition can further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations can comprise microcapsules, such as hydroxymethylcellulose or gelatin-microcapsules, liposomes, albumin microspheres, microemulsions, nanoparticles or nanocapsules.

A topical formulation can be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams can be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, can also contain solvents, emulsifiers, moisturizers, emollients, fragrances, dyes/colorants, preservatives and other active ingredients that increase or enhance the efficacy of the final product. Liquids are thinner than creams, lotions, or gels and often do not contain emulsifiers. Liquid topical products often contain solvents, emulsifiers, moisturizers, emollients, fragrances, dyes/colorants, preservatives and other active ingredients that increase or enhance the efficacy of the final product.

Suitable emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or non-ionic gums such as hydroxyethylcellulose, xanthan gum, magnesium aluminum silicate, silica, microcrystalline wax, beeswax, paraffin, and cetyl palmitate. A gel composition can be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate can be used within topical formulations.

Suitable preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colors include, but are not limited to, FD&C Red No. 40 and FD&C Yellow No. 5. Other suitable additional ingredients that can be included in a topical formulation include, but are not limited to, abrasives, absorbents, anti-caking agents, anti-foaming agents, anti-static agents, astringents (e.g., witch hazel, alcohol and herbal extracts such as chamomile extract), binders/excipients, buffering agents, chelating agents, film forming agents, conditioning agents, propellants, opacifying agents, pH adjusters and protectants.

Modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used, and compositions can be formulated for transdermal administration as a transdermal patch.

A pharmaceutical composition can be formulated as inhaled formulations, including sprays, mists, or aerosols. Such formulations are particularly useful for the treatment of asthma or other respiratory conditions. For inhalation formulations, the compounds provided herein can be delivered via any inhalation methods known to those skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method can include propellants, surfactants and co-solvents and can be filled into conventional aerosol containers that are closed by a suitable metering valve.

Inhalant compositions can comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the desired dosage amount of the liquid composition to be inhaled into the subject's lungs. Suitable formulations, wherein the carrier is a liquid, for administration, as for example, a nasal spray or as nasal drops, include aqueous or oily solutions of the active ingredient.

Formulations or compositions suitable for nasal administration, wherein the carrier is a solid, include a coarse powder having a particle size, for example, in the range of 20 to 500 microns which is administered in the manner in which snuff is administered by rapid inhalation through the nasal passage from a container of the powder held close up to the nose). Suitable powder compositions include, by way of illustration, powdered preparations of the active ingredient thoroughly intermixed with lactose or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation.

Pharmaceutical compositions can also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions can be formulated as sustained release formulations (i.e., a formulation such as a capsule that effects a slow release of modulator following administration). Such formulations can generally be prepared using well known technology and administered by, for example, oral, rectal or subcutaneous implantation, or by implantation at the desired target site. Carriers for use within such formulations are biocompatible, and can also be biodegradable; in some embodiments the formulation provides a relatively constant level of modulator release. The amount of modulator contained within a sustained release formulation can be based upon, for example, the site of implantation, the rate and expected duration of release and the nature of the condition to be treated or prevented.

For the prevention and/or treatment of B₂-bradykinin receptor mediated angioedema (e.g., HAE), the dose of the biologically active compound disclosed herein can vary within wide limits and can be adjusted to individual requirements. Active compounds (compounds of formula (I) or (II), such as, 1-((4-chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile) described herein are generally administered in a therapeutically effective amount. Doses can range from about 0.2 mg to about 50 mg of a compound having formula (I) or (II)/active compound per kilogram body weight, about 0.2 mg to about 35 mg per kilogram body weight, about 0.2 mg to about 20 mg per kilogram of body weight, or about 0.2 mg to about 14.4 mg per kilogram of body weight and can be repeatedly administered every from about 5 hours to about 12 hours, about 10 hours to about 12 hours, or between about 2 and about 5 times per day or between about 2 and about 3 times per day. The daily dose can be administered as a single dose or in a plurality of doses. The amount of active ingredient that can be combined with the carrier materials to produce a single dosage form can be based upon the subject treated and the particular mode of administration. Dosage unit forms will generally contain between from about 0.5 mg to about 100 mg, about 0.5 mg to about 20 mg, about 0.5 to about 10 mg, or about 0.6 mg to about 6 mg of an active ingredient.

It will be understood, however, that the specific dose level for any particular subject can be adjusted based upon a variety of factors including the activity of the specific compound employed, the age, body weight, general health, sex, diet, time of administration, route of administration, and rate of excretion, drug combination (i.e., other drugs being used to treat the subject) and the severity of the particular disease undergoing therapy.

Active compounds disclosed herein will have certain pharmacological properties. Such properties include, but are not limited to oral bioavailability, such that the oral dosage forms discussed above can provide therapeutically effective levels of the compound in vivo.

8-(arylmethoxy)quinolines and 8-(heteroarylmethoxy)quinolines provided herein can be used as antagonists of B₂-bradykinin receptors. B₂-bradykinin receptor antagonists according to embodiments can be used to inhibit the binding of B₂-bradykinin receptor ligands (e.g., bradykinin (BK)) to B₂-bradykinin receptor in vitro or in vivo. B₂-bradykinin receptor modulator(s) provided herein can be administered to a subject (e.g., a human) orally or sublingually, and are present within at least one body fluid or tissue of the subject while modulating B₂-bradykinin receptor activity.

B₂-bradykinin receptor modulators can be useful for the treatment and/or prevention and/or prophylaxis of B₂-bradykinin receptor mediated angioedema, such as hereditary angioedema (HAE). Embodiments including compounds having formula (I) or (II) or salts, stereoisomer, hydrates or solvates thereof can be used as or for the manufacture of a diagnostic agent, whereby such diagnostic agent is for the diagnosis of B₂-bradykinin receptor mediated angioedema.

Compounds of embodiments can be labeled by isotopes, fluorescence or luminescence markers, antibodies or antibody fragments, any other affinity label like nanobodies, aptamers, peptides, etc., enzymes or enzyme substrates. These labeled compounds can be useful for mapping the location of bradykinin receptors in vivo, ex vivo, in vitro and in situ (e.g., in tissue sections via autoradiography) and as radiotracers for positron emission tomography (PET) imaging, single photon emission computerized tomography (SPECT) and the like to characterize those receptors in living subjects or other materials.

Embodiments also pertain to methods for altering the signal-transducing activity of bradykinin receptors in vitro and in vivo. For instance, compounds of certain embodiments and labeled derivatives thereof can be used as standard and reagent in determining the ability of a potential pharmaceutical to bind to the B₂-bradykinin receptor.

Some embodiments can provide methods for localizing or detecting a B₂-bradykinin receptor in a tissue (e.g., a tissue section), which methods involve contacting the tissue sample containing B₂-bradykinin receptor with a detectably labeled compound according to embodiments under conditions that permit binding of the compound to the B₂-bradykinin receptor and detecting the bound compound. Such methods and their respective conditions are known to those skilled in the art and include, for example, radioligand binding assays.

Some embodiments can provide methods of inhibiting the binding of bradykinin (BK) or any other B₂-bradykinin receptor ligand to a B₂-bradykinin receptor which methods involve contacting a solution containing a B₂-bradykinin receptor antagonist compound disclosed herein with cells expressing B₂-bradykinin receptor under conditions and in an amount sufficient to detectably inhibit binding of BK or any other substance to B₂-bradykinin receptor. Such methods and their respective conditions are known to those skilled in the art and include, for example, calcium mobilization assays.

Certain embodiments can provide methods for treating subjects suffering from B₂-bradykinin receptor mediated angioedema as mentioned above. As used herein, the term “treatment” encompasses both disease-modifying treatment and symptomatic treatment, either of which can be prophylactic (i.e., before the onset of symptoms, in order to prevent, delay or reduce the severity of symptoms) or therapeutic (i.e., after the onset of symptoms, in order to reduce the severity and/or duration of symptoms). A B₂-bradykinin receptor mediated angioedema is “responsive to B₂-bradykinin receptor modulation” if modulation of B₂-bradykinin receptor activity results in alleviation of the condition or a symptom thereof. Subjects can include but are not limited to primates (especially humans), domesticated companion animals (such as dogs, cats, horses) and livestock (such as cattle, pigs, sheep), with dosages as described herein.

The compounds of formula (I) or (II) according to embodiments can have improved properties when compared to B₂-bradykinin receptor antagonists known in the state of the art, especially, improved selectivity, low toxicity, low drug-drug interaction, improved bioavailability (especially with regard to oral administration), improved metabolic stability, improved stability in microsomal degradation assay, and improved solubility.

8-(heteroarylmethoxy)quinolines provided herein can be used as agonists or antagonists of B₂-bradykinin receptors in a variety of applications, both in vitro and in vivo. B₂-bradykinin receptor antagonists according to certain embodiments can be used to inhibit the binding of B₂-bradykinin receptor ligands (e.g., BK) to B₂-bradykinin receptor in vitro or in vivo. B₂-bradykinin receptor modulator(s) provided herein can be administered to a subject (e.g., a human) orally or topically, and can be present within at least one body fluid or tissue of the subject while modulating B₂-bradykinin receptor activity.

The following Examples further define and describe embodiments herein. Unless otherwise indicated, all parts and percentages are by weight.

### Examples

The compounds of described embodiments can be prepared in a number of ways well known to one skilled in the art of organic synthesis. The compounds of embodiments can be synthesized using synthetic methods known in the art of synthetic organic chemistry, or variations thereon as appreciated by those skilled in the art.

The compounds shown in the following Table 1 are representative examples of compounds of formula (I) or (II) of embodiments. The CID (Chemical Identification number) listed in the table below can be used to retrieve chemical and biological information available regarding a given compound in the PubChem database at pubchem.ncbi.nlm.nih.gov. Compounds in Table 1 have been shown to at least have binding/antagonist activity with regard to the B₂-bradykinin receptor and information relating to biological activity of the compounds can be found herein and in the PubChem database (based on the CID). The compounds are share a similar core structure (e.g., formula (I))

TABLE 1

| Ex- | | | | Cita- |
|---|---|---|---|---|
| am- | | | Weight | tion/ |
| ple | Structure | Name | (Da) | CID |
| 1 | 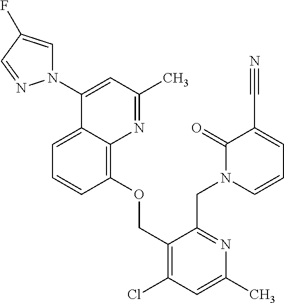 [SMILES: Cc1cc(Cl)c(COc2cccc3c(-n4cc(F)cn4)cc(C)nc23)c(Cn2cccc(C#N)c2=O)n1] | 1-((4-Chloro-3-(((4-(4-fluoro-1H- pyrazol-1-yl)-2-methylquinolin-8- yl)oxy)methyl)-6-methylpyridin-2- yl)methyl)-2-oxo-1,2- dihydropyridine-3-carbonitrile | 514.9 | 25017677 |
| 2 | 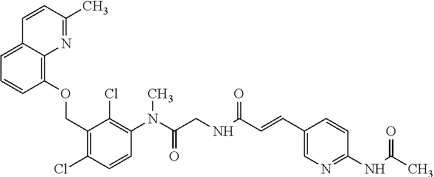 [SMILES: CC(=O)Nc1ccc(/C=C/C(=O)NCC(=O)N(C)c2ccc(Cl)c(COc3cccc4ccc(C)nc34)c2Cl)cn1] | (2E)-3-[6-(Acetylamino)pyridin-3- yl]-N-{2-[(2,4-dichloro-3-{[(2- methylquinolin-8- yl)oxy]methyl}phenyl)(methyl)amino]- 2-oxoethyl}prop-2-enamide | 592.5 | 5311108 |
| 3 | 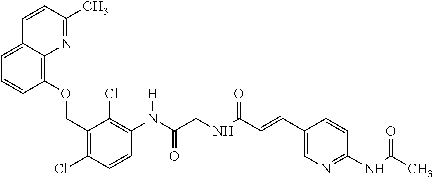 [SMILES: CC(=O)Nc1ccc(/C=C/C(=O)NCC(=O)Nc2ccc(Cl)c(COc3cccc4ccc(C)nc34)c2Cl)cn1] | (2E)-3-[6-(Acetylamino)pyridin-3- yl]-N-{2-[(2,4-dichloro-3-{[(2- methylquinolin-8- yl)oxy]methyl}phenyl)amino]-2- oxoethyl}prop-2-enamide | 578.5 | 10555202 |
| 4 | 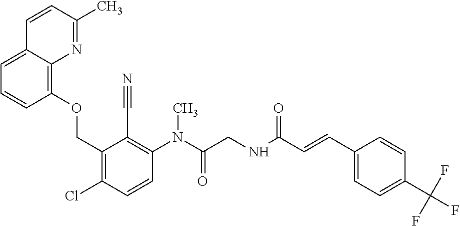 [SMILES: Cc1ccc2cccc(OCc3c(Cl)ccc(N(C)C(=O)CNC(=O)/C=C/c4ccc(C(F)(F)F)cc4)c3C#N)c2n1] | (2E)-N-{2-[(4-Chloro-2-cyano-3- {[(2-methylquinolin-8- yl)oxy]methyl}phenyl)(methyl)amino]- 2-oxoethyl}-3-[4- (trifluoromethyl)phenyl]prop-2- enamide | 593.0 | 22008895 |
| 5 | 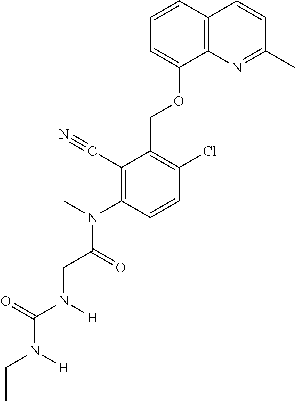 [SMILES: CCNC(=O)NCC(=O)N(C)c1ccc(Cl)c(COc2cccc3ccc(C)nc23)c1C#N] | N-[4-chloro-2-cyano-3-[(2- methylquinolin-8- yl)oxymethyl]phenyl}-2- (ethylcarbamoylamino)-N- methylacetamide | 465.9 | 10895913 |
| 6 | 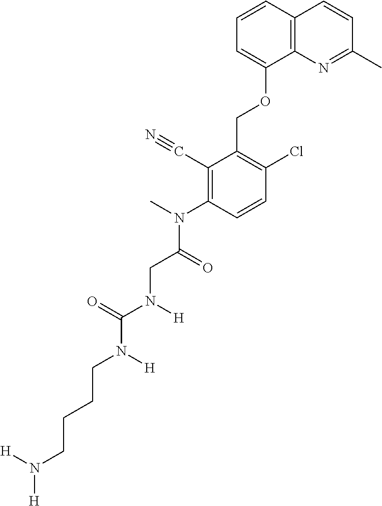 [SMILES: Cc1ccc2cccc(OCc3c(Cl)ccc(N(C)C(=O)CNC(=O)NCCCCN)c3C#N)c2n1] | 2-(4-aminobutylcarbamoylamino)-N- [4-chloro-2-cyano-3-[(2- methylquinolin- 8-yl)oxymethyl]phenyl]-N- methylacetamide | 509.0 | 9831859 |
| 7 | 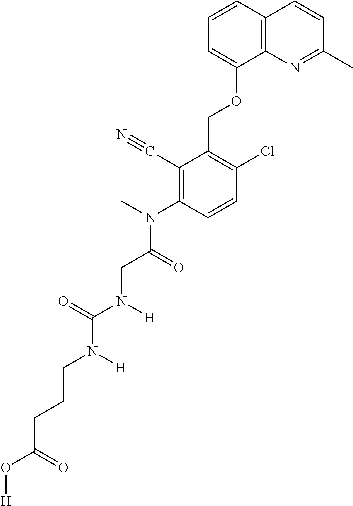 [SMILES: Cc1ccc2cccc(OCc3c(Cl)ccc(N(C)C(=O)CNC(=O)NCCCC(=O)O)c3C#N)c2n1] | 4-[[2-[4-chloro-2-cyano-N-methyl-3- [(2-methylquinolin-8-yl)oxymethyl] anilino]-2- oxoethyl]carbamoylamino]butanoic acid | 524.0 | 44340760 |
| 8 | 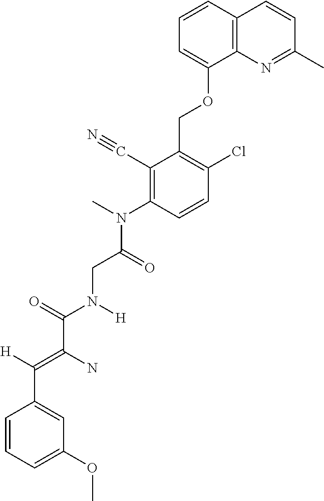 [SMILES: COc1cccc(/C=C(\N)C(=O)NCC(=O)N(C)c2ccc(Cl)c(COc3cccc4ccc(C)nc34)c2C#N)c1] | (E)-N-[2-[4-chloro-2-cyano-N- methyl-3-[(2-methylquinolin-8- yl)oxymethyl] anilino]-2-oxoethyl]-3-(3- methoxyphenyl)prop-2-enamide | 555.0 | 10257108 |
| 9 | 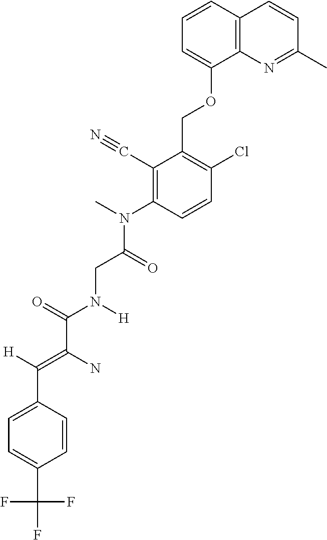 [SMILES: Cc1ccc2cccc(OCc3c(Cl)ccc(N(C)C(=O)CNC(=O)/C(N)=C/c4ccc(C(F)(F)F)cc4)c3C#N)c2n1] | (E)-N-[2-[4-chloro-2-cyano-N- methyl-3-[(2-methylquinolin-8- yl)oxymethyl] anilino]-2-oxoethyl]-3-[4- (trifluoromethyl)phenyl]prop-2- enamide | 593.0 | 22008895 |
| 10 | 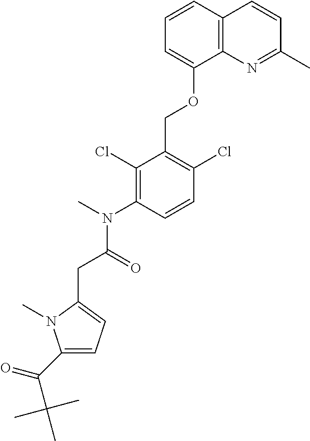 [SMILES: Cc1ccc2cccc(OCc3c(Cl)ccc(N(C)C(=O)Cc4ccc(C(=O)C(C)(C)C)n4C)c3Cl)c2n1] | N-[2,4-dichloro-3-[(2- methylquinolin-8- yl)oxymethyl]phenyl]-2-[5-(2, 2-dimethylpropanoyl)-1- methylpyrrolo-2-yl]-N- methylacetamide | 552.5 | 44276753 |
| 11 | 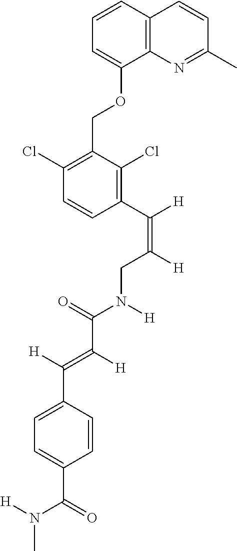 [SMILES: CNC(=O)c1ccc(/C=C/C(=O)NC/C=C\c2ccc(Cl)c(COc3cccc4ccc(C)nc34)c2Cl)cc1] | 4-[(E)-3-[[(Z)-3-[2,4-dichloro-3-[(2- methylquinolin-8- yl)oxymethyl]phenyl]prop-2- enyl]amino]-3-oxoprop-1-enyl]-N- methylbenzamide | 560.5 | 11800992 |
| 12 | 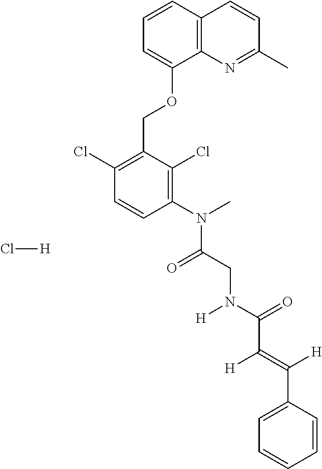 [SMILES: Cl ; Cc1ccc2cccc(OCc3c(Cl)ccc(N(C)C(=O)CNC(=O)/C=C/c4ccccc4)c3Cl)c2n1] | (E)-N-[2-[2,4-dichloro-N-methyl-3- [(2-methylquinolin-8- yl)oxymethyl]anilino]-2- oxoethyl]-3-phenylprop-2-enamide hydrochloride | 570.9 | 22113233 |
| 13 | 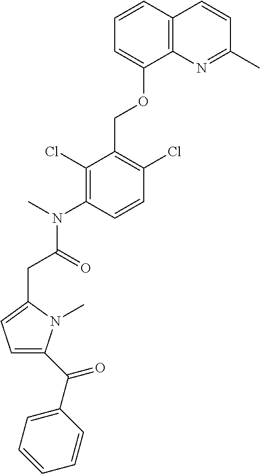 [SMILES: Cc1ccc2cccc(OCc3c(Cl)ccc(N(C)C(=O)Cc4ccc(C(=O)c5ccccc5)n4C)c3Cl)c2n1] | 2-(5-benzoyl-1-methylpyrrol-2-yl)-N- [2,4-dichloro-3-[(2-methylquinolin-8- yl)oxymethyl]phenyl]-N- methylacetamide | 572.5 | 44276711 |
| 14 | 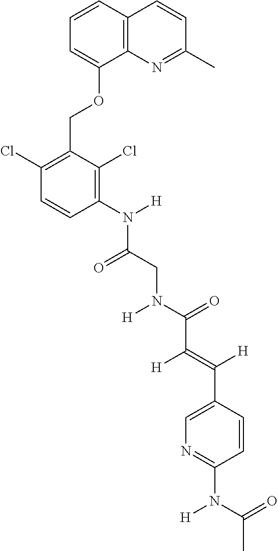 [SMILES: CC(=O)Nc1ccc(/C=C/C(=O)NCC(=O)Nc2ccc(Cl)c(COc3cccc4ccc(C)nc34)c2Cl)cn1] | (E)-3-(6-acetamidopyridin-3-yl)-N- [2-[2,4-dichloro-3-[(2- methylquinolin-8- yl)oxymethyl]anilino]-2- oxoethyl]prop- 2-enamide | 578.4 | 10555202 |
| 15 | 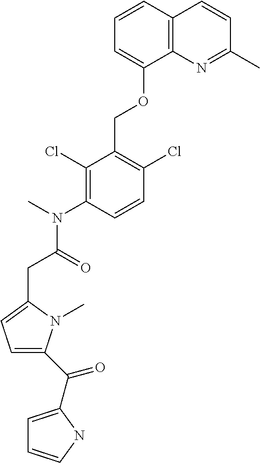 [SMILES: Cc1ccc2cccc(OCc3c(Cl)ccc(N(C)C(=O)Cc4ccc(C(=O)c5ccc[nH]5)n4C)c3Cl)c2n1] | N-[2,4-dichloro-3-[(2- methylquinolin-8- yl)oxymethyl]phenyl]-N-methyl-2-[1- methyl-5-(thiophene-2- carbonyl)pyrrol-2-yl]acetamide | 578.5 | 44276380 |
| 16 | 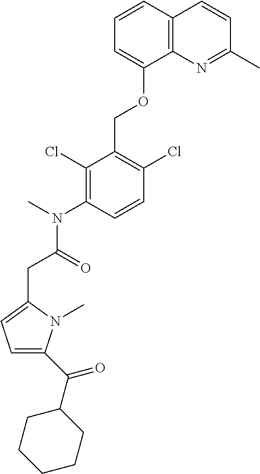 [SMILES: Cc1ccc2cccc(OCc3c(Cl)ccc(N(C)C(=O)Cc4ccc(C(=O)C5CCCCC5)n4C)c3Cl)c2n1] | 2-[5-(cyclohexanecarbonyl-1- methylpyrrol-2-yl]-N-[2,4-dichloro-3- [(2-methylquinolin-8- yl)oxymethyl]phenyl]-N- methylacetamide | 578.5 | 44276287 |
| 17 | 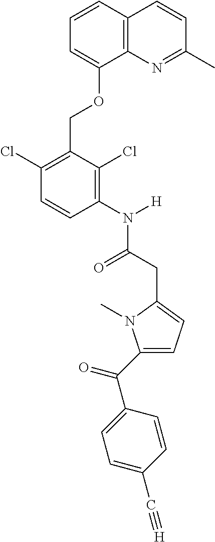 | 2-[5-(4-cyanobenzyl)-1- methylpyrrol-2-yl]-N-[2,4-dichloro-3- [(2-methylquinolin-8- yl)oxymethyl]phenyl]acetamide | 583.5 | 44276487 |
| 18 | 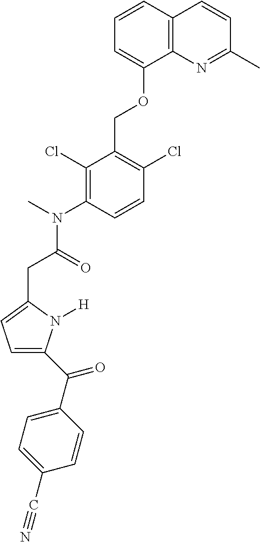 [SMILES: Cc1ccc2cccc(OCc3c(Cl)ccc(N(C)C(=O)Cc4ccc(C(=O)c5ccc(C#N)cc5)[nH]4)c3Cl)c2n1] | 2-[5-(4-cyanobenzyl)-1H-pyrrol-2- yl]-N-[2,4-dichloro-3-[(2- methylquinolin-8- yl)oxymethyl]phenyl]-N- methylacetamide | 583.5 | 22288581 |
| 19 | 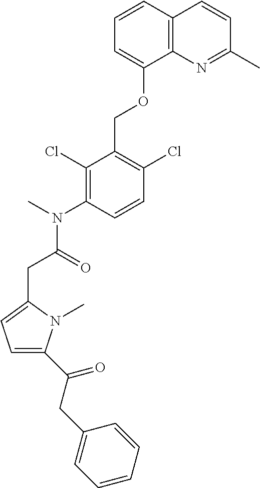 [SMILES: Cc1ccc2cccc(OCc3c(Cl)ccc(N(C)C(=O)Cc4ccc(C(=O)Cc5ccccc5)n4C)c3Cl)c2n1] | N-[2,4-dichloro-3-[(2- methylquinolin-8- yl)oxymethyl]phenyl]-N-methyl-2-[1- methyl-5-(2-phenylacetyl)pyrrol-2- yl]acetamide | 586.5 | 44276375 |
| 20 | 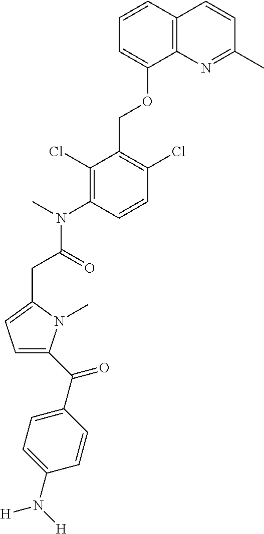 [SMILES: Cc1ccc2cccc(OCc3c(Cl)ccc(N(C)C(=O)Cc4ccc(C(=O)c5ccc(N)cc5)n4C)c3Cl)c2n1] | 2-[5-(4-aminobenzoyl)-1- methylpyrrol-2-yl]-N-[2,4-dichloro-3- [(2-methylquinolin-8- yl)oxymethyl]phenyl]-N- methylacetamide | 587.5 | 22288562 |
| 21 | 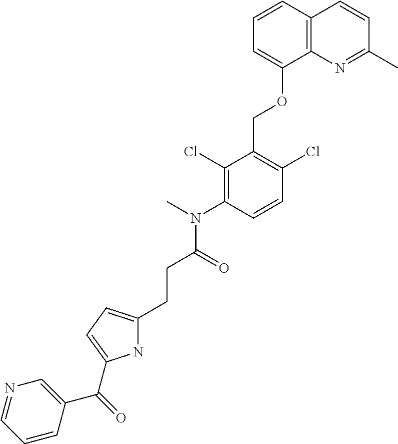 [SMILES: Cc1ccc2cccc(OCc3c(Cl)ccc(N(C)C(=O)CCc4ccc(C(=O)c5cccnc5)[nH]4)c3Cl)c2n1] | N-[2,4-dichloro-3-[(2- methylquinolin-8- yl)oxymethyl]phenyl]-N-methyl-3-[1- methyl-5-(pyridine-3- carbonyl)pyrrol-2-yl]propanamide | 587.5 | 4426793 |
| 22 | 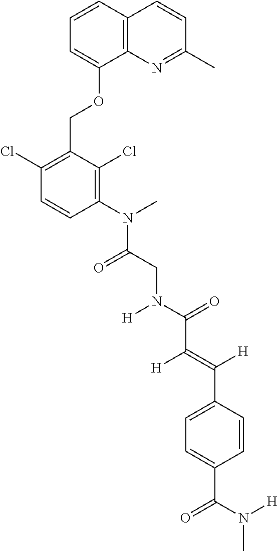 [SMILES: CNC(=O)c1ccc(/C=C/C(=O)NCC(=O)N(C)c2ccc(Cl)c(COc3cccc4ccc(C)nc34)c2Cl)cc1] | 4-[(E)-3-[[2-[2,4-dichloro-N-methyl- 3-[(2-methylquinolin-8- yl)oxymethyl]anilino]-2- oxoethyl]amino]-3-oxoprop-1-enyl]- N-methylbenzamide | 591.5 | 10348414 |
| 23 | 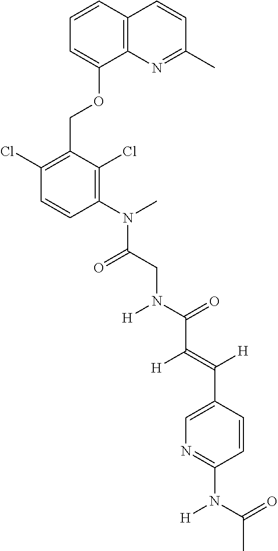 [SMILES: CC(=O)Nc1ccc(/C=C/C(=O)NCC(=O)N(C)c2ccc(Cl)c(COc3cccc4ccc(C)nc34)c2Cl)cn1] | (E)-3-(6-acetamidopyridin-3-yl)-N- [2-[2,4-dichloro-N-methyl-3-[(2- methylquinolin-8- yl)oxymethyl]anilino]-2- oxoethyl]prop-2-enamide | 592.5 | 5311108 |
| 24 | 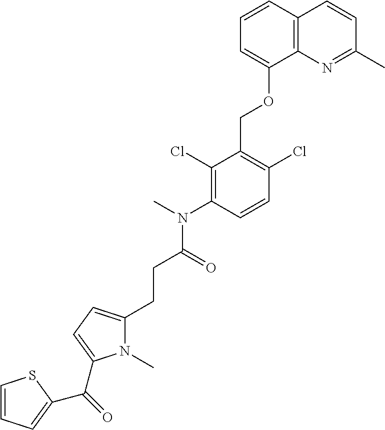 [SMILES: Cc1ccc2cccc(OCc3c(Cl)ccc(N(C)C(=O)CCc4ccc(C(=O)c5cccs5)n4C)c3Cl)c2n1] | N-[2,4-dichloro-3-[(2- methylquinolin-8- yl)oxymethyl]phenyl]-N-methyl-3-[1- methyl-5-(thiophene-2- carbonyl)pyrrol-2-yl]propanamide | 592.5 | 44276474 |
| 25 | 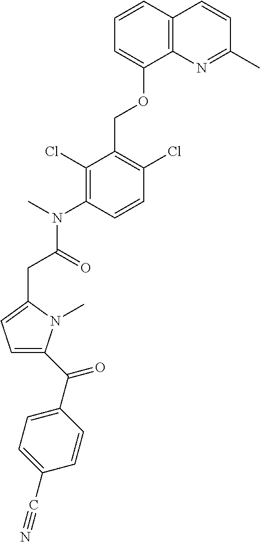 [SMILES: Cc1ccc2cccc(OCc3c(Cl)ccc(N(C)C(=O)Cc4ccc(C(=O)c5ccc(C#N)cc5)n4C)c3Cl)c2n1] | 2-[5-(4-cyanobenzyl)-1- methylpyrrol-2-yl]-N-[2,4-dichloro-3- [(2-methylquinolin-8- yl)oxymethyl]phenyl]-N- methylacetamide | 597.5 | 15952860 |
| 26 | 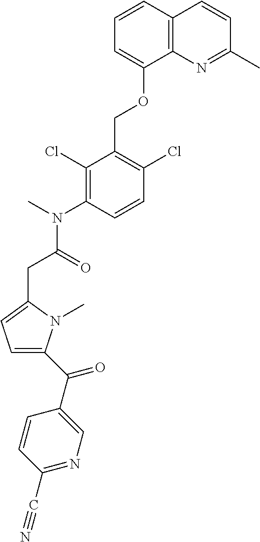 [SMILES: Cc1ccc2cccc(OCc3c(Cl)ccc(N(C)C(=O)Cc4ccc(C(=O)c5ccc(C#N)nc5)n4C)c3Cl)c2n1] | 2-[5-(6-cyanopyridine-3-carbonyl)-1- methylpyrrol-2-yl]-N-[2,4-dichloro-3- [(2-methylquinolin-8- yl)oxymethyl]phenyl]-N- methylacetamide | 598.5 | 44276488 |
| 27 | 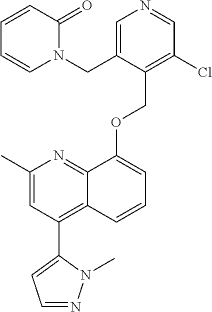 [SMILES: Cc1cc(-c2ccnn2C)c2cccc(OCc3c(Cl)cncc3Cn3ccccc3=O)c2n1] | 1-[[5-chloro-4-[[2-methyl-4-(2- methylpyrazol-3-yl)quinolin-8-yl] oxomethyl]pyridin-3- yl]methyl]pyridin-2-one | 471.9 | 44190942 |
| 28 | 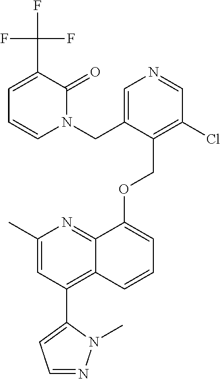 [SMILES: Cc1cc(-c2ccnn2C)c2cccc(OCc3c(Cl)cncc3Cn3cccc(C(F)(F)F)c3=O)c2n1] | 1-[[5-chloro-4-[[2-methyl-4-(2- methylpyrazol-3-yl)quinolin-8-yl] oxymethyl]pyridin-3-yl]methyl]-3- (trifluoromethyl)pyridin-2-one | 539.9 | 44190944 |

### Example 29

### Synthesis of 1-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile

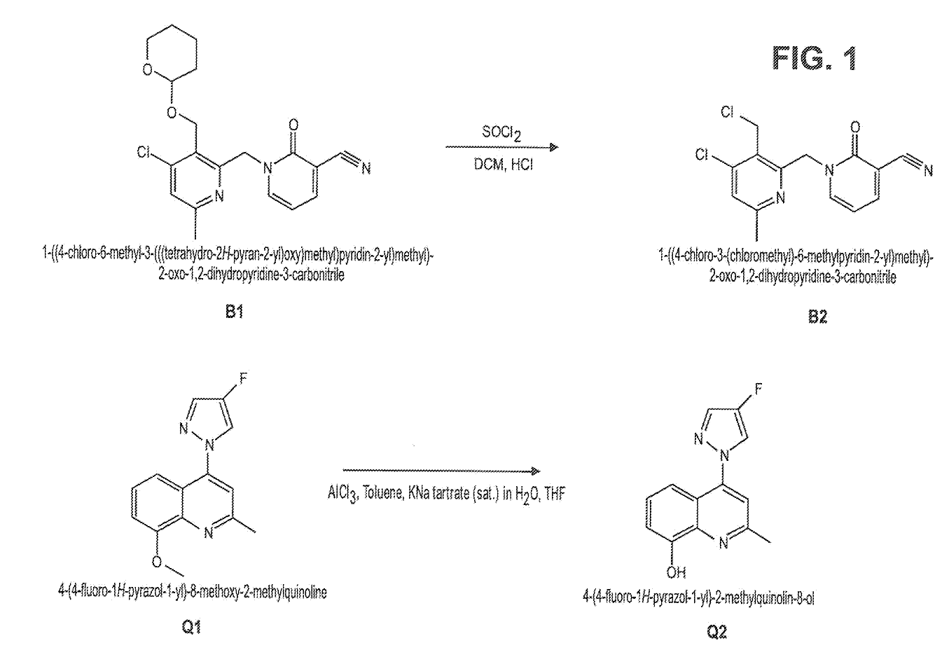

1-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (CFMQ) was synthesized using starting materials B1 and Q1 shown in FIG. 1. In reaction step 1,1-((4-chloro-6-methyl-3-(((tetrahydro-2H-pyran-2-yl)oxy)methyl)pyridine-2-yl)methyl)-2-oxo-1,2-dihydropyridine-3-carbonitrile (B1) was deprotected under acidic conditions in the presence of thionyl chloride to remove the tetrahydropyranyl ether protecting group and replace the alcohol with a chlorine (C1) atom forming 1-((4-chloro-3-(chloromethyl)-6-methylpyridin-2-yl)methyl)-2-oxo-1,2-dihydropyridine-3-carbonitrile (B2). In reaction step 2, the methyl ether protecting group on 4-(4-fluoro-1H-pyrazol-1-yl)-8-methoxy-2-methylquinoline (Q1) was cleaved by aluminum chloride (AlCl₃) in toluene leaving the reactive hydroxyquinoline, 4-(4-fluoro-1H-pyrazol-1-yl)-2-methylquinolin-8-ol (Q2). In the final step (reaction step 3), B2 and Q2 are reacted in the presence of potassium carbonate in acetonitrile/water to form an ether linkage between the reactive hydroxyl and chloromethyl groups resulting in the CFMQ.

### Example 30

### Physicochemical Properties of CFMQ

The physicochemical properties of 1-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile (CFMQ) were determined using standard methods. See Tables 2 and 3, below. CFMQ was a free base with a molecular weight of 514.9 Da and was a nonhygroscopic, crystalline powder with a melting point of about 214° C. While CFMQ had slight solubility in aqueous medium, its solubility was greatly enhanced in acidic environments.

TABLE 2
Summary of Physicochemical Properties of CFMQ

| | |
|---|---|
| Molecular Weight | 514.9 |
| Solubility (mM) in phosphate buffered | 33 |
| saline (PBS) | |
| pKa1 | 3.95 |
| pKa2 | 1.70 |
| logD (pH = 7.4) | 3.47 |
| Total Polar Surface Area (A²) | 98.6 |
| Number of Rotatable Hoods | 6 |
| H-bond Acceptors | 8 |
| H-bond Donors | 0 |

TABLE 3
Summary of Solubility Properties of CFMQ

| Vehicle | pH | Solubility (mg/ml) |
|---|---|---|
| Water | — | <0.01 |
| HCl 0.1N | 1.3 | 0.32 |
| Citrate/NaOH pH 2 | 2 | 0.02 |
| Citrate/NaOH pH 5 | 5 | <0.01 |
| Phosphate Buffer | 7 | <0.01 |
| NaOH 0.1N | 12.8 | <0.01 |
| Acetic acid, glacial | — | 19.89 |
| Ethanol | — | 0.17 |
| Glycerol | — | 0.01 |
| PEG400 (polyethylene | — | 2.37 |
| glycol 400) | | |
| Propylene Glycol | — | 0.10 |
| Labrasol (glyceryl and | — | 1.43 |
| polyethylene glycol esters) | | |
| Olive Oil | — | <0.01 |
| Tween 20 (Polyoxyethylene | — | 0.87 |
| (20) sorbitan monolaurate) | | |
| Tween 80 (Polyoxyethylene | — | 3.50 |
| (80) sorbitan monolaurate) | | |

### Example 31

### Efficacy of CFMQ

In vivo experiments in both wild-type (WT) C57BL/6J mice and C1-inhibited knock out (C1 INH KO) mice measured pharmacodynamic efficacy by the ability of CFMQ to block plasma extravasation, which is causative to edema, the most important symptom requiring treatment in HAE subjects. This plasma extravasation model has been used extensively in the literature to investigate the efficacy of bradykinin receptor antagonists. (Han et al., “Increased vascular permeability in C1 inhibitor-deficient mice mediated by the bradykinin type 2 receptor,” J Clin Invest, 2002 April; 109(8): 1057-1063.)

Determination of ED₅₀ in Mice

Intravenous Study in Wild-Type Mice

The uptake and effective blocking of plasma extravasation of CFMQ was examined following IV (intravenous) bolus administration in wild-type (WT) C57/BL6 male mice (n=4/dose group). Mice received CFMQ (HGT3711) (2 μL/g) at 1.0 μg/kg, 100 μg/kg, and/or 6 mg/kg. Following CFMQ administration and prior to the terminal time point, mice received an IV injection of Evans blue (EB) dye (30 mg/kg) and were sacrificed. The bladder was removed, dried and weighed and extracted in formamide (1.0 mL). EB concentration in the formamide extract was determined spectrophotometrically and EB content was calculated as μg EB per milligram of tissue weight. Efficacy was determined by inhibiting the accumulation of EB in the bladder.

Doses of 1.0 and or 100 μg/kg did not inhibit extravasation, showing similar levels of EB in the bladder as controls, with the 1.0 μg/kg dose giving a value of 0.7602 EB mg/kg tissue, compared to controls (0.7927 EB mg/kg tissue). However, the 6 mg/kg dose achieved significant inhibition of extravasation compared to the control group, with a value of 0.0701 EB mg/kg tissue.

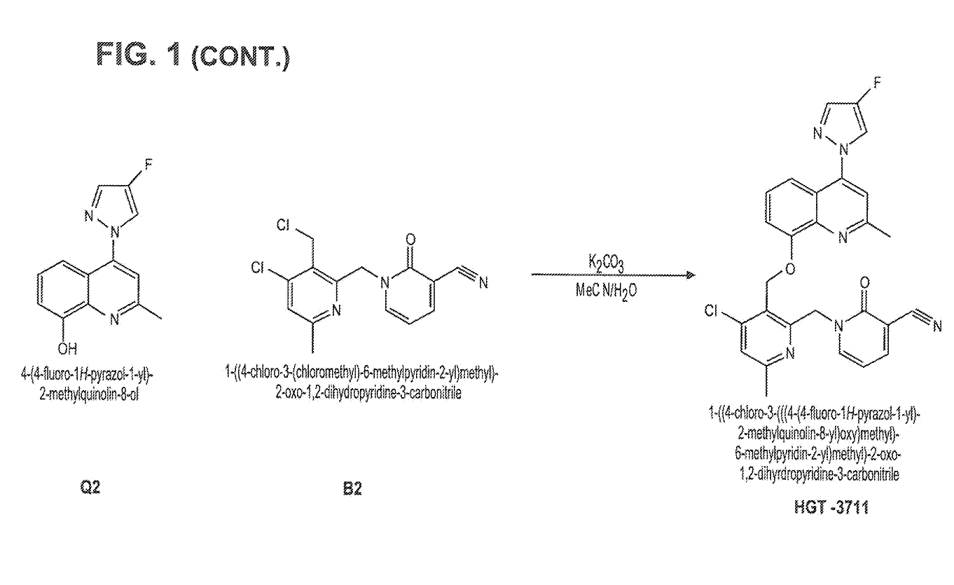

CFMQ doses of 0.001, 0.05, 0.1, 0.25, 0.5, 1.0, and or 3.0 mg/kg were administered as IV bolus injections to WT mice (n=8/dose group). As illustrated in FIG. 2, a dose-response compared to vehicle controls was demonstrated, with significant inhibition of plasma extravasation at doses of 0.5, 1.0, and or 3.0 mg/kg. When dosed intravenously in the WT model, CFMQ (HGT3711) blocked plasma extravasation completely at high doses (500 μg/kg) and showed an effective dose (ED₅₀) of 460 μg/kg (FIG. 2).

Oral Administration Study in Wild-Type Mice

The uptake and effective blocking of plasma extravasation of CFMQ (HGT3711) was examined following oral administration in wild-type (WT) C57/BL6 male mice (n=4/dose group). Mice received a single oral gavage of CFMQ (HGT3711) (10 μL/g) at 3.0 or 10 mg/kg. Blood samples were taken at 20 and 60 minutes post-dose. Following oral administration CFMQ (HGT3711) administration and prior to the terminal time point, mice received an IV injection of Evans blue (EB) dye (30 mg/kg) and were sacrificed. Similar to the IV study, EB concentration in the formamide extract was determined spectrophotometrically (μg EB per milligram of tissue) and efficacy was determined by inhibiting the accumulation of EB in the bladder.

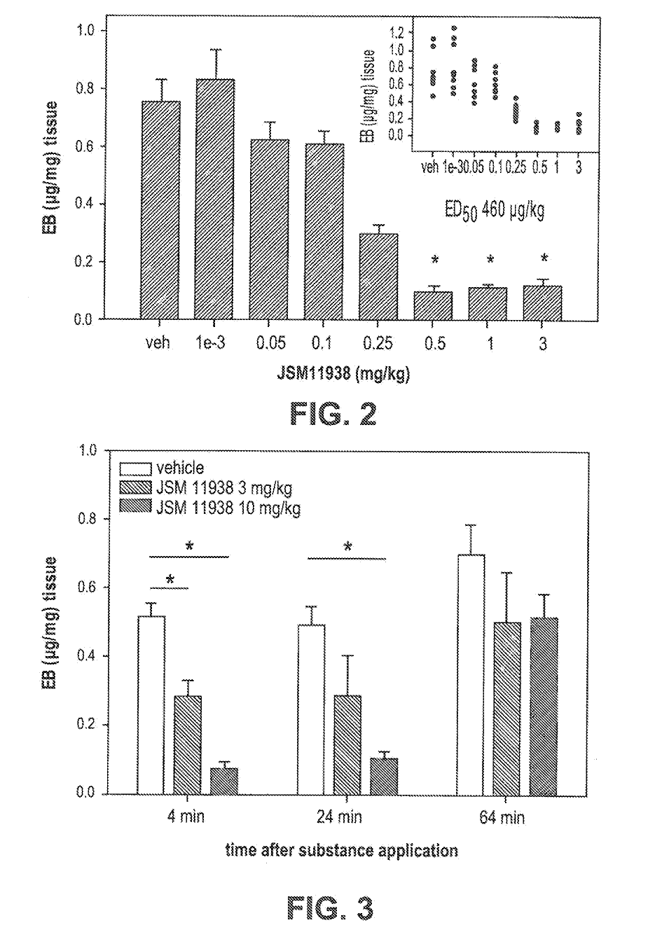

As illustrated in FIG. 3, both doses of CFMQ (HGT3711) had an inhibitory effect on plasma extravasation, with significant differences seen at 10 and 30 mg/kg (4 minutes) and at 10 mg/kg (24 minutes) post-dose, compared to vehicle controls. At 3.0 mg/kg inhibition to 56% and 58% at 4 and 24 minutes post dose were observed. A dose of 10 mg/kg showed greater inhibition of extravasation to 14% and 21% at 4 and 24 minutes, respectively. After 64 minutes, inhibition was at 72% and 75% for the 3.0 and 10 mg/kg doses, respectively. These results supported that oral administration of CFMQ (HGT3711) demonstrated rapid pharmacodynamic effects in the mouse.

The efficacy of CFMQ (HGT3711) following orally administration was studied in female WT mice. Eight mice per group received either vehicle or CFMQ (HGT3711) at 1.0, 3.0, 10, or 30 mg/kg by oral gavage. An additional dose group received Firazyr® (icatibant-0.4 mg/kg) as a subcutaneous (SC) injection. Similar to the previous studies, inhibition of EB content in the dried bladder (μg EB per milligram of tissue weight) was utilized as measurement of efficacy.

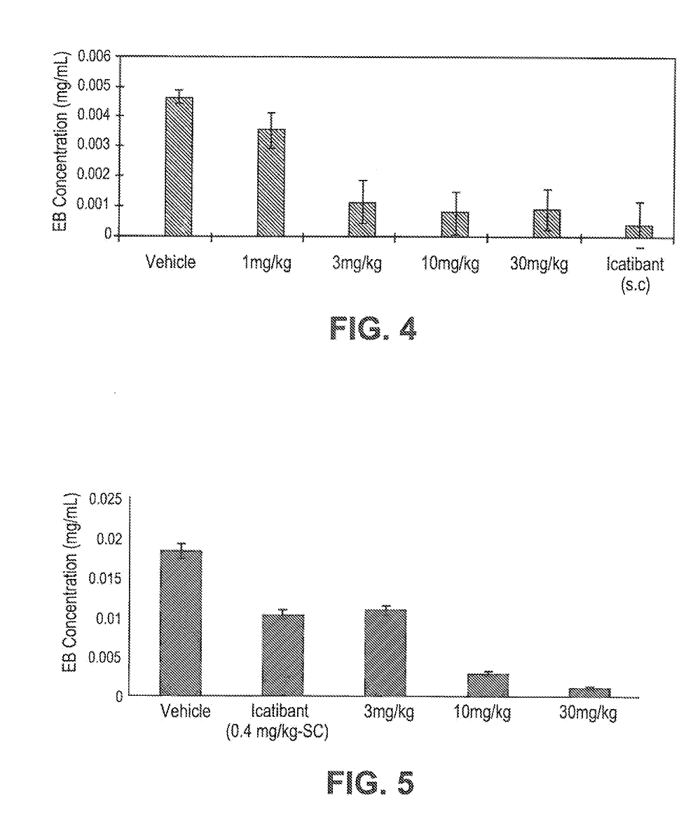

The EB concentration of the formamide extract from the bladder demonstrated CFMQ (HGT3711) inhibition of EB absorbance in a dose dependent manner. An oral dose of 3.0 mg/kg was determined to be the minimally-effective dose (MED), and a dose of 10.0 mg/kg (plasma value of 267 nM) was found to be equally effective as a 0.4 mg/kg SC dose of Firazyr® (icatibant). The results illustrated in FIG. 4 show that both 10 and 30 mg/kg doses of CFMQ (HGT3711) provided almost 100% inhibition of EB accumulation in the bladder.

Oral Administration Study in Knockout Mice

To demonstrate efficacy in a mouse model relevant to the HAE disease, studies were conducted in a knockout mouse that is deficient in the C-1 inhibitor (C1-INH-KO). These mice contain a similar genetic deficit to the HAE patient/subject population and demonstrated increased vascular permeability as compared to wild-type littermates.

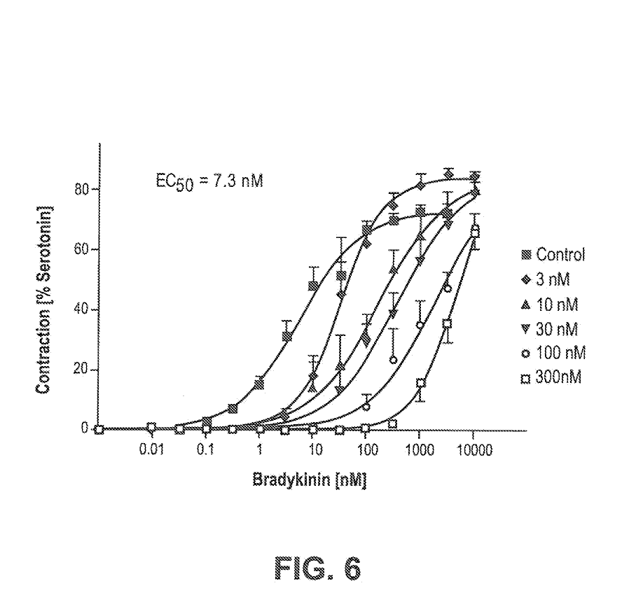

In a similar study design as described above, oral administration of CFMQ (HGT3711) demonstrated a similar efficacy profile as observed in the WT mice, with dose-dependent inhibition of EB dye extravasation in the bladder of CFMQ (HGT3711) dosed animals (FIG. 5).

The EB concentration of the formamide extract from the bladder of knockout mice demonstrated CFMQ inhibition of EB absorbance in a dose dependent manner. An oral dose of 3.0 mg/kg was determined to be nearly as effective as a 0.4 mg/kg SC dose of Firazyr® (icatibant) in the knockout mice and both 10 and 30 mg/kg doses of CFMQ provided almost 100% inhibition of EB accumulation in the bladder of knockout mice.

### Example 32

### Potency of 1-((4-Chloro-3-(((4-(4-fluoro-H-pyrazol-1-yl)-2-methylquinolin-8-oxy)methyl)-6-methylpyridin-2-yl)methyl)-2-oxo-1,2-dihydropyridine-3-carbonitrile

The potency of CFMQ (JSM11938/HGT3711) was determined in B₂-bradykinin receptor binding and functional cellular and ex vivo assays. CFMQ was potent in assays that assessed binding to the B₂-bradykinin receptor and functional assays that measured calcium mobilization as a marker of B₂-bradykinin receptor binding.

Receptor Binding and Selectivity

To determine the affinity to the B₂-bradykinin receptor, CFMQ was compared to a diverse set of receptors and reference agents in an in vitro assay using cells from rat heart, urinary bladder, cerebral cortex, as well as human recombinant cells (CHO and HEK 293) and other cell lines. Each assay included a ligand at a specific concentration (concentration ranging from 0.007-10 nM), in addition to a non-specific ligand (concentration ranging from 0.1 μM-50 nM) for incubation periods ranging from 15 minutes to 6 hours at 4°-37° C. The specific binding to the receptors was defined as the difference between the total binding and the nonspecific binding, determined in the presence of an excess of unlabeled CFMQ. The concentration causing a half-maximal inhibition of control specific binding (IC₅₀ values) and inhibition constants (Kᵢ) were determined, and each reference compound was within the accepted limits of the historic average (±0.5 log units).

CFMQ was found to bind to a small number of off-target (non-bradykinin 2) receptors with IC₅₀ values less than 10 μM (Table 4). The levels at which the off-target receptors were bound were greater than ten times higher than the concentrations required to influence efficacy. CFMQ (HGT3711) was demonstrated to be selective in its binding to the B₂-bradykinin receptor and to have a strong binding affinity to the B₂-bradykinin receptor.

TABLE 4
Receptors ≧50% Inhibited by CFMQ at 10 μM

| | | IC₅₀ | Kᵢ | |
|---|---|---|---|---|
| Receptor | % inhibition | (μM) | (μM) | nH |
| μ | 54 | 6.7 | 2.7 | 1.0 |
| (h) (MOP) (agonist site) | | | | |
| A₃ | 53 | 7.6 | 4.5 | 1.4 |
| (h) | | | | |
| BZD | 93 | 1.4 | 1.2 | 1.7 |
| (peripheral) | | | | |
| NK₃ | 53 | 1.1 | 2.7 | 1.1 |
| (h) | | | | |
| V₁ₐ | 51 | 1.5 | 9.4 | 1.3 |
| (h) | | | | |

In a competition assay, CFMQ was formulated with incubation buffer (containing 2 nM [³H]bradykinin) and brought to concentrations of 0.001, 0.003, 0.01, 0.03, 0.1, 0.3, 1.0 and 3.0 μM. For determination of non-specific binding 1.25 μL of a 10 mM bradykinin (BK) solution was added to 248.8 μL incubation buffer (containing 2 nM [³H]BK). For determination of total binding, CFMQ was not added. Appropriate controls were prepared in the same method.

Human embryonic kidney (HEK) 293 cells stably express recombinant human B₂-bradykinin receptors (10 pmol/mg protein) and were added to 96-well culture-trays and cultivated for 1-3 days, followed by incubation with 100 μL of each of the incubation buffers containing [³H]BK. After a 90-minute incubation period and washing (4×PBS (phosphate buffered saline)) supernatants of the cell mixtures were transferred to scintillation vials and assayed for [³H]BK in a beta-counter. Results of counts per minute (cpm) for non-specific binding were subtracted from the total cpm and were used for curve fit and IC₅₀ calculation.

Results showed that CFMQ (HGT3711) bound to and competed away bradykinin with an IC₅₀ of 3.3 nM. The reference compound Firazyr® (icatibant) exhibited similar affinity for B₂-bradykinin receptor with an IC₅₀ of 2 nM.

The results of these studies demonstrated that CFMQ (HGT3711) has selective and strong binding affinity to the B₂-bradykinin receptor.

Calcium Mobilization Assays

CFMQ activity on the inhibition of calcium mobilization, a marker of B₂-bradykinin receptor binding was characterized in a cellular assay. CFMQ was formulated as a 5 nM stock solution in 100% DMSO and serially diluted to 0.04, 0.12, 0.37, 1.11, 3.33, 10 and 30 nM. Human fibroblast (HF15) cells, which express the human B2R were loaded with 100 μL calcium dye solution containing 2.5 mM probenicide and were then pre-incubated with CFMQ for 25 minutes at 25° C. The inhibition effect of CFMQ on bradykinin-mediated calcium mobilization was tested in this system by emission of fluorescence signals, using the height of the resulting peak over the baseline value (relative fluorescence units [RFU] max-min). The percent inhibition for each concentration was used for curve fit and IC₅₀ calculation.

CFMQ was found to have a strong potency to the human B2-bradykinin receptor where it inhibited bradykinin-induced calcium mobilization with an IC₅₀ of 2.97 nM. In a previous study the reference compound Firazyr® (icatibant) exhibited an IC₅₀ of 4.0 nM. (Data not shown.)

Umbilical Vein Contraction Assays

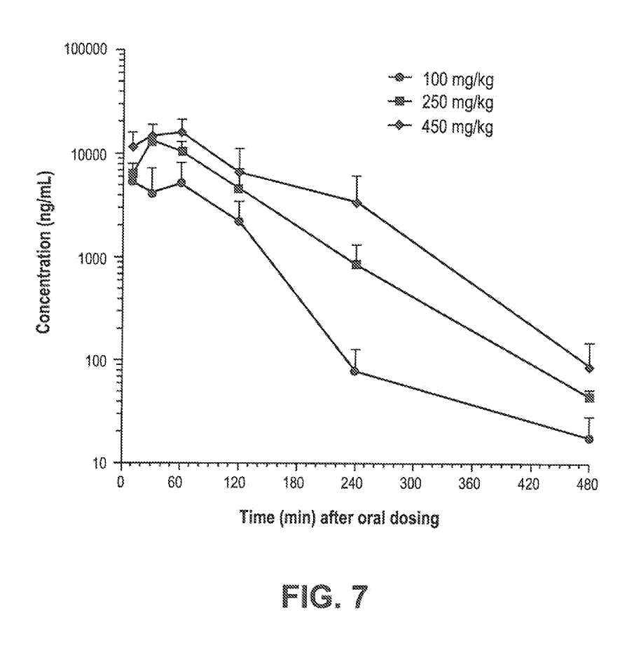

The inhibition effect of CFMQ on bradykinin induced calcium mobilization was examined in an ex vivo functional assay of human umbilical vein contraction, which is considered a gold standard for bradykinin activity measurements. The human umbilical cord preparation was comprised of a control condition (no bradykinin agonist), CFMQ at 10, 30, 100 and 300 nM concentrations and a positive control group with reference to a known B2R antagonist (icatibant; Firazyr®). Following a 30 minute incubation, BK-induced vein contractions were initiated in a cumulative manner (final concentration of 10 μM), followed by maximal calibration contraction induction by 10 μM serotonin. The tension increase for each dose response was calculated in relation to the (maximal) response towards serotonin, graphed as a dose-response curve and used to calculate an effective concentration at 50% (EC₅₀) value (FIG. 6)

In this functional umbilical vein assay, an EC₅₀ of 7.3 nM for CFMQ (FIG. 6) was found to be comparable to Firazyr® (icatibant), which had an EC₅₀ of 2.5 nM.

Permeability

To determine bidirectional permeability, CFMQ was examined in an in vitro incubation assay with human small intestinal mucosa (Caco-2 cell). CFMQ at 5 μM was dosed to the cell monolayers on the apical side (A-to-B) or basolateral side (B-to-A) and incubated at 37° C. with 5% CO₂ for 120 minutes. Permeability of Lucifer Yellow (500 μM) was measured to ensure no damage was inflicted to the cell monolayers during the CFMQ flux period. All samples were assayed by LC-MS/MS using electrospray ionization.

The result was a permeability coefficient of 34 to 38 cm/s (A-to-B; B-to-A) respectively, which was considered highly permeable with no significant efflux in the Caco-2 cells. This absorption in humans was not expected to be permeability limited, and was a favorable predictor for both rapid absorption and time to onset of action of an orally administered molecule.

### Example 33

### Pharmacokinetics of CFMQ in Mouse, Rat, Dog, Monkey and Micro Yucatan Miniature Swine

Absorption in Mice

CFMQ (HGT3711) was administered to fasted female CD-1 mice (n=18/dose group) by a single oral gavage at 100, 250 and 450 mg/kg in a dose volume of 5 mL/kg in a spray-dried dispersion formulation (discussed below). Blood samples were taken via cardiocentesis or abdominal vein prior to administration and at 10, 30, 60 (1 hr), 120 (2 hr), 240 (4 hr) and 480 (8 hr) minutes post dose. Plasma concentrations of CFMQ (HGT3711) were determined by LC-MS/MS (liquid chromatography mass spectrometry) and concentrations below the limit of quantitation (3 ng/mL) were assigned a value of zero for pharmacokinetic analysis. Nominal dosing concentrations were used in all calculations.

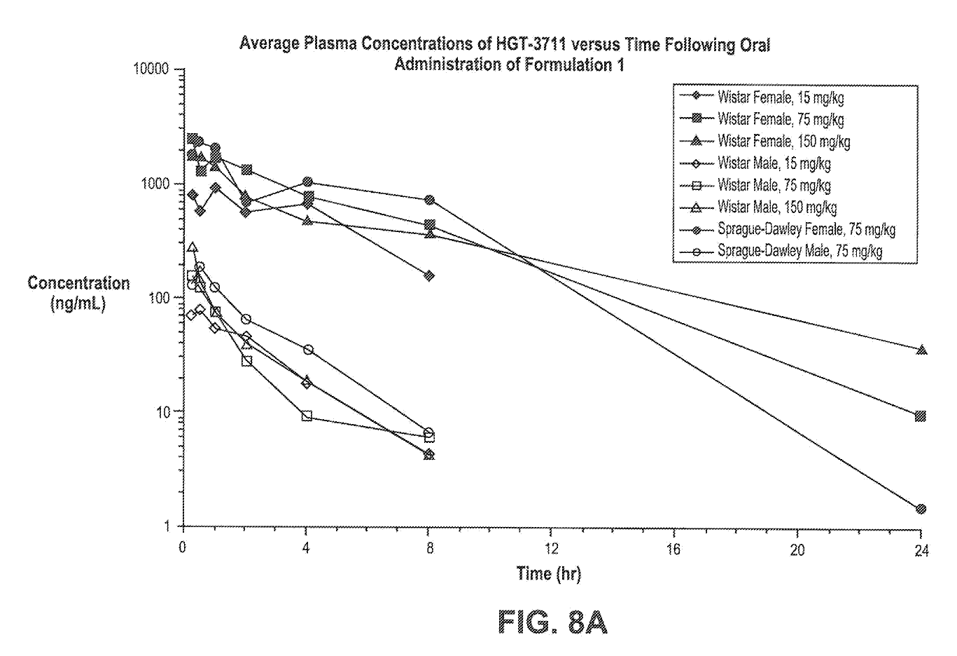

FIG. 7 illustrates the time-concentration curve at each dose level and Table 5 summarizes the plasma concentration (ng/mL) and PK (pharmacokinetic) properties of CFMQ (HGT3711) following oral administration in mice.

TABLE 5
Average Plasma Concentration (ng/mL) and PK Properties of
CFMQ (HGT3711) Following IV Administration in CD-1 Mice
Average Concentration of HGT3711 (ng/mL) ± SD
at Sampling Time points (min)

| Dose | 10 | 30 | 60 | 120 | 240 | 480 |
|---|---|---|---|---|---|---|
| (mg/kg) | min | min | min | min | min | min |
| 100 | 5297 ± | 4100 ± | 5170 ± | 2200 ± | 78 ± | 18 ± |
| | 1520 | 3014 | 2856 | 1246 | 48 | 11 |
| 250 | 6250 ± | 13267 ± | 10403 ± | 4570 ± | 854 ± | 45 ± |
| | 1580 | 451 | 2685 | 2544 | 468 | 7 |
| 450 | 11573 ± | 14633 ± | 15750 ± | 6435 ± | 3374 ± | 88 ± |
| | 4252 | 4046 | 5445* | 4660* | 2691 | 63 |

Pharmacokinetic Properties

| Dose | Cₘₐₓ | AUCₗₐₛₜ | AUC_{∞} |
|---|---|---|---|
| (mg/kg) | (ng/mL) | (min · ng/mL) | (min · ng/mL) |
| 100 | 5297.0 | 628805.0 | 630160.0 |
| 250 | 13267.0 | 1463980.0 | 1467486.0 |
| 450 | 15750.0 | 2445200.0 | 2452475.0 |

ND: Not Determined;
¹Extrapolated to t = 0;
*n = 2 animals per time point

There was a dose proportional increase in the exposure profile of the mouse following oral administration of CFMQ (HGT3711). Absorption was rapid with the time of maximum plasma concentration (Tₘₐₓ) values at or near the first time point collected. The peak serum concentration (Cₘₐₓ) was seen to compress as the doses got larger, however the exposure (area under the concentration curve; AUC_{∞}) continued to increase proportionally. This may reflect a maximum in absorption rate at doses higher than 250 mg/kg with this formulation.

Absorption in Rats

Pharmacokinetics and gender differences were evaluated with 2 different formulations of CFMQ (HGT3711) in 2 different types of rat models. These attributes were investigated in a single oral gavage study. Male and female Wistar rats (n=3 per dose group) received CFMQ (HGT3711) at 15, 75 75, and or 150 mg/kg. Male Sprague-Dawley (S-D) rats (n=3 per dose group) received a single dose level, 75 mg/kg. The two vehicle formulations evaluated were formulation 1: 10% N-methyl pyrrolidone (NMP): 10% Ethanol: 30% polyethylene glycol (PEG400): 50% GELUCIRE (lauroyl polyoxylglycerides); and formulation 2: 10% NMP: 0% PEG400: 20% CREMOPHOR EL (Macrogolglycerol ricinoleate): 25% GELUCIRE 44/14 (lauroyl polyoxylglycerides): 25% CAPROYOL 90 (Propylene Glycol Caprylate).

Blood samples were taken via cardiac puncture prior to administration and at 0.25 (15 min), 0.50 (30 min), 1, 2, 4, 8 and 24 hours post dose. Plasma concentrations of CFMQ (HGT3711) were determined by LC-MS/MS and nominal dosing concentrations were used in all calculations.

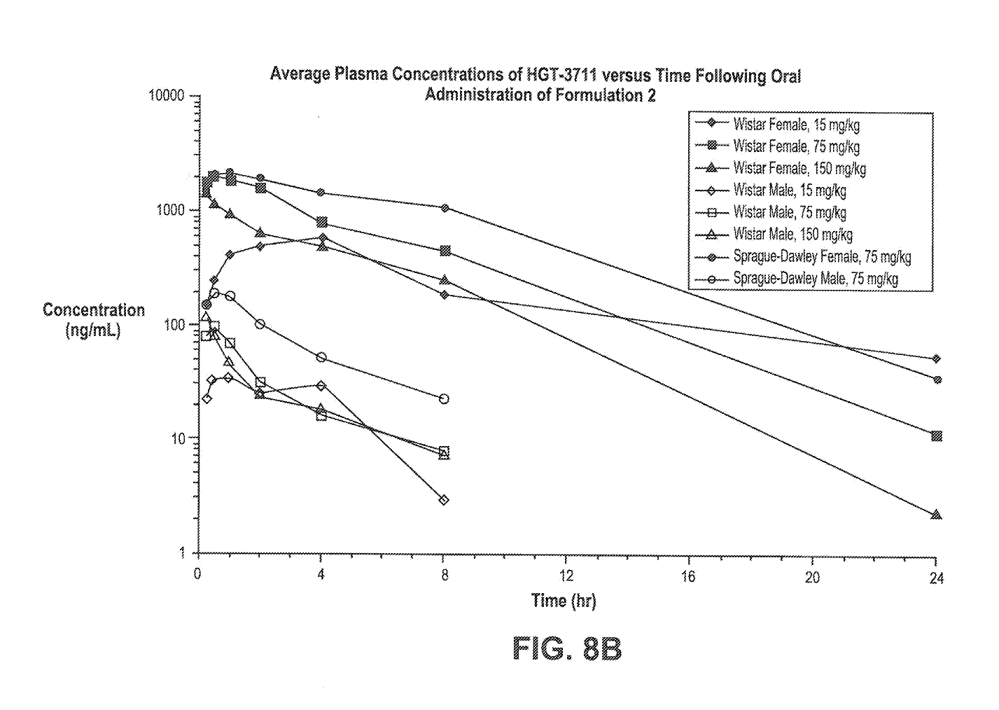

No adverse reactions were observed in any rats in this study. FIG. 8 illustrates the time-concentration curve of both formulations in both sexes of each species.

Based on average dose normalized AUC values and similar dose concentration, oral exposure to CFMQ (HGT3711) was 20-40 times higher in female rats compared to male rats of both species (Table 6). This gender difference was observed in both S-D and Wistar rats. In both dose formulations in the Wistar rat, the average AUC values decreased with increasing dose, showing that CFMQ (HGT3711) exposure was not dose proportional; however, this was likely due to precipitation of the molecule at the higher concentrations.

TABLE 6
Average Pharmacokinetic Properties of CFMQ (HGT3711)
Following Oral Administration of 2 Formulations in
Male and Female Wistar and Sprague Dawley Rats

| | | | | | | |
|---|---|---|---|---|---|---|
| | | | | | AUCₗₐₛₜ | AUC_{∞} |
| Strain | Dose | Cₘₐₓ | Tₘₐₓ | t_{1/2} | (hr · | (hr · |
| (Sex) | (mg/kg) | (ng/mL) | (hr) | (hr) | ng/mL) | ng/mL) |

Formulation 1

| | | | | | | |
|---|---|---|---|---|---|---|
| Wistar | 15 | 1138 | 0.83 | 3.10 | 4662 | 6647 |
| (Female) | 75 | 2547 | 0.58 | 3.13 | 11085 | 11130 |
| | 150 | 1853 | 0.33 | 5.42 | 8712 | 9059 |
| Wistar | 15 | 77.4 | 0.50 | 1.67 | 218 | 229 |
| (Male) | 75 | 155 | 0.25 | 4.28 | 243 | 282 |
| | 150 | 277 | 0.25 | 1.63 | 309 | 397 |
| S-D (Female) | 75 | 2277 | 0.50 | 2.01 | 11935 | 14271 |
| S-D (Male) | 75 | 187 | 0.50 | 1.96 | 410 | 435 |

Formulation 2

| | | | | | | |
|---|---|---|---|---|---|---|
| Wistar | 15 | 592 | 3.33 | 6.08 | 5210 | 5814 |
| (Female) | 75 | 2197 | 0.75 | 3.12 | 10738 | 11317 |
| | 150 | 1580 | 0.33 | 2.64 | 6427 | 6436 |
| Wistar | 15 | 34.7 | 0.83 | 1.74 | 175 | 155 |
| (Male) | 75 | 94.9 | 0.50 | 3.35 | 219 | 261 |
| | 150 | 119 | 0.25 | 2.43 | 199 | 260 |
| S-D (Female) | 75 | 2177 | 0.83 | 3.58 | 20604 | 20842 |
| S-D (Male) | 75 | 191 | 0.67 | 3.43 | 673 | 745 |

Within both formulations, CFMQ (HGT3711) demonstrated large differences in exposure between sexes, with females showing greater than a 5-fold increase in exposure compared to males. The vehicle formulation utilized in the studies was a spray-dried dispersion formulation of CFMQ (HGT3711) complexed with 50% hydroxyl propyl methyl cellulose acetate succinate (HPMCAS) (discussed below).

Absorption in Micro Yucatan Miniature Swine

CFMQ (HGT3711) was administered to female Yucatan Miniature Swine (mini-pig; n=3) by a single oral gavage at 10 mg/kg (concentration 2 mg/mL) at a dose volume of 5.0 mg/mL. This study utilized the spray-dried dispersion (SDD) (50; 50 with polymer; HPMCAS) formulation of CFMQ (HGT3711) (see below). Blood samples were taken via jugular or other suitable vein puncture prior to administration and at 0.083 (5 min), 0.25 (15 min), 0.50 (30 min), 1, 2, 4, 8, 12 and 24 hours post dose. Plasma concentrations of CFMQ (HGT3711) were determined by LC-MS/MS.

There were no test articles related to mortality/morbidity and no abnormal clinical observations noted during the course of the study.

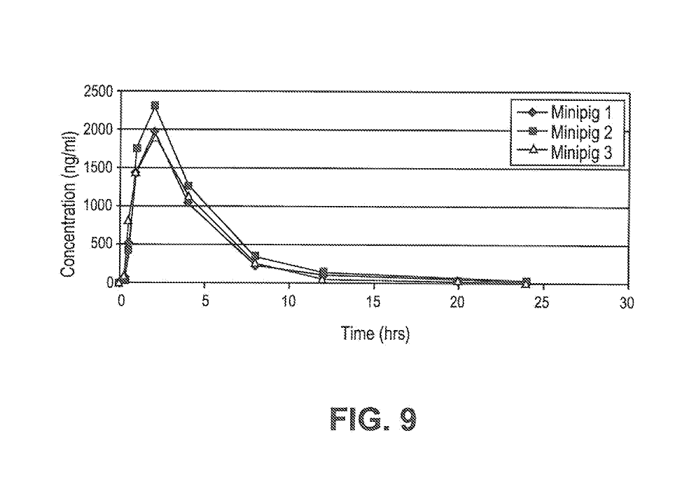

The average plasma concentration time curve is seen in FIG. 9 and pharmacokinetic properties and plasma concentrations of CFMQ (HGT3711) are summarized in Table 7.

TABLE 7
Average Plasma Concentration (ng/mL) and PK Profile of
CFMQ (HGT3711) Following PO Administration in Female
Yucatan mini-pig at 10 mg/kg
Average Concentration of HGT3711 (ng/mL) at Sampling
Time points (hr)

| 0 | 0.25 | 0.5 | 1.0 | 2.0 | 4.0 | 8.0 | 12 | 24 |
|---|---|---|---|---|---|---|---|---|
| (hr) | (hr) | (hr) | (hr) | (hr) | (hr) | (hr) | (hr) | (hr) |
| 0 | 80.0 | 595.7 | 1556.7 | 2063.3 | 1153.3 | 283.7 | 111.5 | 14.2 |

Pharmacokinetic Properties

| Tₘₐₓ | t_{1/2} | CL | Vₛₛ | AUCₗₐₛₜ | AUC_{∞} |
|---|---|---|---|---|---|
| (hr) | (hr) | (L/hr/kg) | (L/kg) | (hr · ng/mL) | (hr · ng/mL) |
| ND | 57 | ND | ND | 10625 | 11115 |

ND: Not Determined;
¹Extrapolated to t = 0.

Following oral administration in mini-pig, averaged plasma levels of CFMQ (HGT3711) had a half-life of 57 hours and an extrapolated AUC of 11115 hr·ng/mL.

Plasma Kinetics Following Intravenous Exposure in Mice

CFMQ (HGT3711) was administered to fasted female CD-1 mice by a single intravenous injection at 1.0 mg/kg, formulated in 100% PEG200. Blood samples were taken via cardiac puncture prior to administration and at 0.083 (5 min), 0.25 (15 min), 0.50 (30 min), 1, 2, 4, and 8 hours post dose. Plasma concentrations of CFMQ (HGT3711) were determined by LC-MS/MS and concentrations below the limit of quantitation (BLOQ=<1 ng/ml) were assigned a value of zero for pharmacokinetic analysis. Nominal dosing concentrations were used in all calculations.

No adverse reactions were observed after the IV (intravenous) administration CFMQ (HGT3711). Table 8 summarizes the average plasma concentration and calculated pharmacokinetic properties of CFMQ in the mouse. By 4 hours post-dose CFMQ (HGT3711) plasma concentration was below the BLOQ.

TABLE 8
Average Plasma Concentration (ng/mL) and PK Properties
of CFMQ (HGT3711) Following IV Administration
in Female CD-1 Mice at 1 mg/kg
Average Concentration of HGT3711 (ng/mL) ± SD
at Sampling Time points (hr)

| 0 | 0.083 | 0.25 | 0.50 | 1.0 | 2.0 | 4.0 | 8.0 |
|---|---|---|---|---|---|---|---|
| ND | 162 ± | 92.9 ± | 22.4 ± | 12.6 ± | 3.48 ± | ND | ND |
| | 76.6 | 70.1 | 1.84 | 8.59 | ND | | |

Pharmacokinetic Properties

| | | | | | AUCₗₐₛₜ | |
|---|---|---|---|---|---|---|
| C₀ | Tₘₐₓ | t_{1/2} | CL | Vₛₛ | (hr · ng/ | AUC_{∞} |
| (ng/mL)¹ | (hr)¹ | (hr) | (L/hr/kg) | (L/kg) | mL) ± SE | (hr · ng/mL) |
| 214 | 0 | 0.56 | 14.1 | 6.3 | 68.1 ± | 70.9 |
| | | | | | 10.8 | |

ND: Not Determined;
¹Extrapolated to t = 0

Following IV administration, averaged plasma levels of CFMQ (HGT3711) had a half-life (t_{1/2}) of 0.56 hours and a clearance (CL) rate of 14.1 L/hr/kg, which is greater than liver blood flow in a typical mouse (5.40 L/hr/kg). (Davies and Morris, “Physiological parameters in laboratory animals and humans,” Pharm Res, 1993 July; 10(7):1093-5.) The average volume of distribution (Vss) was 6.3 L/kg, which is greater than total body water in mouse (0.73 L/kg). This suggested that CFMQ (HGT3711) was extensively distributed in the mouse.

Plasma Kinetics Following Intravenous Exposure in Rats

CFMQ (HGT3711) was administered to fasted female Sprague-Dawley rats by a single intravenous injection at 1.0 mg/kg, formulated in 100% PEG200 (Table 2.6.5.X, Study 10SHIRUSP11). Blood samples were taken via a jugular vein cannula prior to administration and at 0.083 (5 min), 0.25 (15 min), 0.50 (30 min), 1, 2, 4 and 8 hours post dose. Plasma concentrations of CFMQ (HGT3711) were determined by LC-MS/MS and concentrations below the limit of quantitation (1 ng/mL) were assigned a value of zero for pharmacokinetic analysis. Nominal dosing concentrations were used in all calculations.

There were no adverse reactions observed after the intravenous administration of CFMQ (HGT3711). Table 9 summarizes the average plasma concentration and calculated pharmacokinetic properties of CFMQ in the rat.

TABLE 9
Average Plasma Concentration (ng/mL) and PK Properties
of CFMQ (HGT3711) Following IV Administration
in Female Sprague-Dawley Rats at 1 mg/kg
Average Concentration of HGT3711 (ng/mL) ± SD
at Sampling Time points (hr)

| 0 | 0.083 | 0.25 | 0.50 | 1.0 | 2.0 | 4.0 | 8.0 |
|---|---|---|---|---|---|---|---|
| hr | hr | hr | hr | hr | hr | hr | hr |
| 0 | 597 ± | 401 ± | 324 ± | 248 ± | 160 ± | 113 ± | 36.9 ± |
| | 351 | 111 | 39.8 | 111 | 58.1 | 73.2 | 46.2 |

Pharmacokinetic Properties (±SD)

| | | | | | AUCₗₐₛₜ | AUC_{∞} |
|---|---|---|---|---|---|---|
| C₀ | Tₘₐₓ | t_{1/2} | CL | Vₛₛ | (hr · | (hr · |
| (ng/mL)¹ | (hr)¹ | (hr) | (L/hr/kg) | (L/kg) | ng/mL) | ng/mL) |
| 747 ± | 0 | 2.86 ± | 0.95 ± | 2.6 ± | 1149 ± | 1413 ± |
| 558 | | 2.54 | 0.68 | 1.1 | 501 | 771 |

Following IV administration in rat, averaged plasma levels of CFMQ (HGT3711) had a half-life of 2.86±2.54 hours and a clearance rate of 0.95 f 0.68 L/hr/kg, which is approximately 30% of liver blood flow in rat (3.3 L/hr/kg) (Davies and Morris, 1993). The average volume of distribution was 2.6±1.1 L/kg, which was high and greater than total body water in rat (0.7 L/kg) (Davies and Morris, 1993) suggesting that CFMQ was extensively distributed in the rat.

Plasma Kinetics Following Intravenous Exposure in Dogs

CFMQ (HGT3711) was administered to fasted female beagle dogs by a single intravenous injection at 1.0 mg/kg, formulated in 100% PEG200 (Table 10). Blood samples were taken via jugular vein puncture prior to administration and at 0.083 (5 min), 0.25 (15 min), 0.50 (30 min), 1, 4, 8 and 24 hours post dose. Plasma concentrations of CFMQ (HGT3711) were determined by LC-MS/MS and concentrations below the limit of quantitation (1.0 ng/mL) were assigned a value of zero for pharmacokinetic analysis. Nominal dosing concentrations were used in all calculations.

TABLE 10
Average Plasma Concentration (ng/mL) and Pharmacokinetic
Properties for CFMQ (HGT3711) Following IV Administration
in Female Beagle Dogs at 1 mg/kg
Average Concentration of HGT3711 (ng/mL) at
Sampling Time points (hr)

| 0 | 0.083 | 0.25 | 0.50 | 1.0 | 4.0 | 8.0 | 24 |
|---|---|---|---|---|---|---|---|
| (hr) | (hr) | (hr) | (hr) | (hr) | (hr) | (hr) | (hr) |
| 0 | 1845 | 1325 | 1070 | 717 | 377 | 180 | ND |

Pharmacokinetic Properties

| | | | | | AUCₗₐₛₜ | AUC_{∞} |
|---|---|---|---|---|---|---|
| C₀ | Tₘₐₓ | t_{1/2} | CL | Vₛₛ | (hr · | (hr · |
| (ng/mL)¹ | (hr)¹ | (hr) | (L/hr/kg) | (L/kg) | ng/mL) | ng/mL) |
| 2175 | 0 | 2.69 | 0.25 | 0.75 | 5155 | 5270 |

ND: Not Determined;
¹Extrapolated to t = 0

Following IV administration in dogs, average plasma levels of CFMQ (HGT3711) demonstrated a half-life of 2.69 hours and clearance rate of 0.25 L/hr/kg, which is approximately 14% of liver blood flow in dog (1.85 L/hr/kg) (Davies and Morris 1993). The average volume of distribution was 0.75 L/kg, similar to total body water in dog (0.6 L/kg), (Davies and Morris 1993). This suggested that CFMQ is not extensively distributed in dogs.

Plasma Kinetics Following Intravenous Exposure in Monkeys

A cross-over PK (pharmacokinetic) analysis was conducted in cynomolgus monkey. Two fasted male monkeys in each dose group received a single 1.0 mg/kg dose of CFMQ (HGT3711) by either IV (intravenous) or PO (oral) administration, with a 7-day washout period between each dose. The dose concentration was 1.0 mg/mL with a dose volume of 1.0 mL/kg. Blood samples were collected via the femoral vein at 0.083 (5 min), 0.25 (15 min), 0.5 (30 min), 1, 1.5, 2, 3, 4, 6, 8, 18 and 24 hours post-dose. Plasma concentrations of CFMQ (HGT3711) were determined by LC-MS/MS and concentrations below the limit of quantitation (2.5 ng/mL) were assigned a value of zero for pharmacokinetic analysis. Following oral administration of CFMQ (1 mg/kg), the concentration of CFMQ in plasma was below the limit of quantitation (BLQ=<2.5 ng/ml) at each sampling time point, preventing assessment of pharmacokinetic properties of CFMQ after oral administration in monkeys in this study. Table 11 summarizes the average PK properties for IV and PO dosed groups.

TABLE 11
Selected Pharmacokinetic Properties of CFMQ (HGT3711) in Male
Cynomolgus Monkeys Following IV and Oral Administration

| | | | | | | | | |
|---|---|---|---|---|---|---|---|---|
| AUC₍₀₋ₜ₎ | ACU_{(0-∞)} | MRT_{(0-∞)} | t_{1/2z} | Tₘₐₓ | Vz | CLz | Cmax | F |
| (μg/L*hr) | (μg/L*hr) | (hr) | (hr) | (hr) | (L/kg) | (L/hr/kg) | (μg/L) | (%) |

IV (1 mg/kg)

| | | | | | | | | | |
|---|---|---|---|---|---|---|---|---|---|
| Mean | 678.74 | 693.99 | 1.77 | 1.40 | 0.23 | 2.98 | 1.51 | 410.17 | N/A |
| SD | 148.70 | 151.48 | 0.23 | 0.24 | 0.20 | 0.55 | 0.41 | 107.14 | N/A |

PO (1 mg/kg)

| | | | | | | | | | |
|---|---|---|---|---|---|---|---|---|---|
| Mean | N/C | N/C | N/C | N/C | N/C | N/C | N/C | N/C | N/C |
| SD | N/C | N/C | N/C | N/C | N/C | N/C | N/C | N/C | N/C |

N/A = not applicable
N/C = not calculated; all plasma levels of HGT3711 at all time point samples were below the limit of detection

Following IV (intravenous) administration in monkeys, the average plasma levels of CFMQ (HGT3711) demonstrated a systemic clearance (CL) of 1.51±0.41 L/hr/kg, which corresponds to 57.63% of monkey hepatic blood flow (2.62 L/hr/kg)(Davies and Morris 1993). The mean half-life (t_{1/2}), Cₘₐₓ and AUC_{(0-∞)} values were 1.40±0.24 hr, 410±107.14 μg/L and 693.99 f 151.48 hr μg/L, respectively. CFMQ administered intravenously distributes extensively into the tissues of the monkey, with a mean volume of distribution (V_{z}) at terminal phase of 2.98±0.55 L/kg, which corresponds to 4.32-fold of the total body water (0.69 L/kg) in the monkey. (Davies and Morris 1993)

A late Tₘₐₓ was observed in Monkey 2 and 4 following intravenous injection, which may have been due to the utilization of 50% PEG200, which has a delayed-response effect and may have made the solution viscous, limiting the solution at the injection site and slowing blood dispersal.

Following oral administration of CFMQ (HGT3711) at 1 mg/kg there was no bioavailability in the monkey, as plasma concentrations of CFMQ were below the limit of quantification (BLQ). However, bioavailability following an oral dose of CFMQ was observed in all other species tested.

Based on in vitro metabolism studies across species the lack of exposure in the monkey may be due to low metabolic stability and/or a unique clearance or transport mechanism in the primate that is not expected in other species, including humans. When CFMQ was dosed to monkeys by the intravenous route it demonstrated moderate to high clearance values, and this may indicate that the lack of bioavailability may in part be due to a lack of absorption. Low absorption could be the result of an interaction with a transporter in the liver or intestine that limits the systemic exposure of CFMQ in monkeys.

Metabolism

The metabolic pathway of CFMQ (HGT3711) was explored in liver microsomes and primary hepatocytes, as well as in an in vivo mini-pig biodistribution study.

In Vitro Metabolism Studies

In vitro metabolite identification was performed in CD-1 mice, Sprague-Dawley rats, Yucatan mini-pigs and human hepatocytes. Observed metabolites were confirmed by comparison against synthetic reference standards.

In vitro metabolic stability studies were performed to determine hepatic stability in vivo. CFMQ was incubated in liver microsomal preparations from mouse, rat, dog, mini-pig, and humans, as well as an additional study with monkey and human. CFMQ was incubated with human and animal liver microsomes at 0.3 mg/mL at 37° C. for 30 or 60 minutes. Additional reference compounds were incubated as controls. Following incubation, samples were analyzed by HPLC-MS/MS.

CFMQ (HGT3711) had variable stability in rodent species with generally increasing stability in higher species, with markedly higher stability in human liver preparations. Low metabolic stability in the monkey corresponded to low bioavailability and may indicate a unique metabolic pathway. See Table 12.

TABLE 12
Summary of In Vitro Microsomal Stability of CFMQ (HGT3711)

| | | % Remaining | % Remaining |
|---|---|---|---|
| Species | Gender | [30 minutes] | [60 minutes] |
| Mouse (CD-1) | Male | 7 | 3 |
| Rat (Wistar) | Male | 3 | 0 |
| Rat (Wistar) | Female | 76 | 60 |
| Dog (Beagle) | Male | 66 | 45 |
| Mini-Pig (Gotting) | Male | 74 | 58 |
| Monkey | Male | 32 | 7 |
| (Cynomolgus) | | | |
| Human | Mix | 70-91 | 50-85 |

Hepatocyte Metabolism

In vitro metabolism of a tritiated labeled form of CFMQ (HGT3711) was assessed in cryopreserved male and female rat and mini-pig hepatocytes, as well as in male and female mouse hepatocytes. Human hepatocytes (mixed-gender) were also examined.

³H-CFMQ1 (concentration of 5 μM or 10 μM; approximately 1.0 mCi/mL) was incubated with 1×10⁶ cells (hepatocytes)/mL hepatocytes for up to four hours. Hepatocytes from all species were characterized for both Phase I and Phase II metabolizing capacity by incubation with positive controls (¹⁴C-7-ethoxycoumarin and ¹⁴C-testosterone) at 2 and 4 hours. At time points of 0.5, 1, 1.5, 2, and 4 hours, incubation samples were extracted by the addition of acetonitrile and analyzed by liquid scintillation (LSC) counting followed by HPLC (high performance liquid chromatography) radiodetection. Selected samples (4 hr) underwent further analysis by LC-MS/MS (liquid chromatography mass spectrometry) to identify those metabolites representing >5% of sample radioactivity.

Within both studies a total of 16 main radioactive regions of interest were detected in incubations of ³H-CFMQ with cryopreserved hepatocytes. Some of these components were multi-component in nature. A summary of the major metabolites observed are listed in Table 13.

TABLE 13
In Vitro Metabolite Identification and Related Species of CFMQ (HGT3711)

| | | | | | | | | | |
|---|---|---|---|---|---|---|---|---|---|
| | Metabolite | Male | Female | Male | Female | Male | Female | Male | Female |
| Component of HGT3711 | ID No^{a} | Mouse | Mouse | Rat | Rat | Mini-pig | Mini-pig | Human | Human |

| | | | | | | | |
|---|---|---|---|---|---|---|---|
| Sulphate of non-hydroxy upper ½ | 1 | ✓ | X | ✓^{#} | ✓^{#} | X | X |
| molecule of HGT3711 | | | | | | | |
| Sulphate of upper ½ molecule of | 2# | ✓ | ✓ | X | ✓ | X | X |
| HGT3711 | | | | | | | |
| Upper ½ molecule of HGT3711 | 2# | ✓ | ✓ | X | ✓ | X | X |
| Glutathione conjugate of mono-hydroxy | 3 | ✓ | X | X | X | X | X |
| de-chlorinated HGT3711 | | | | | | | |
| Not identified | 4 | — | — | — | — | — | — |
| Glutathione conjugate of de-chlorinated | 5# | ✓ | ✓ | ✓ | ✓ | X | X |
| HGT3711 | | | | | | | |
| Glutathione conjugate of mono-hydroxy | 5# | ✓ | ✓ | ✓ | ✓ | X | X |
| de-chlorinated HGT3711 | | | | | | | |
| Glucuronide of di-hydroxy HGT3711 | 6 | ✓ | X | X | X | X | X |
| Glucuronide of mono-hydroxy HGT3711 | 7 | ✓ | ✓ | X | X | X | X |
| Mono-hydroxy HGT3711 | 8# | ✓ | ✓ | ✓ | ✓ | ✓ | ✓ |
| Acid metabolite of HGT3711 | 8# | ✓ | | ✓ | ✓ | ✓ | ✓ |
| Di-hydroxy of HGT3711 | 9 | ✓ | ✓ | ✓ | ✓ | ✓ | ✓ |
| Glucuronide of mono-hydroxy | 10 | ✓ | ✓^{t} | ✓ | ✓ | ✓ | ✓ |
| HGT371112 | | | | | | | |
| Mono-hydroxy HGT3711 | 11 | ✓ | ✓ | ✓ | ✓ | ✓ | ✓ |
| Mono-hydroxy HGT3711 (JSM12609) | 12 | ✓ | ✓ | ✓ | ✓ | ✓ | ✓ |
| Mono-hydroxy HGT3711 (JSM12697) | 13 | ✓ | ✓ | ✓ | ✓ | ✓ | ✓ |
| Mono-hydroxy HGT3711 (JSM12697) | 14 | ✓ | X | ✓^{t} | ✓ | ✓ | ✓ |

^{a}MET numbers were assigned 3H-radiochromatogram of various hepatocyte incubation samples
✓ component detected during LC-MS/MS analyses and >1% of sample radioactivity in 3H-radiochromatogram
✓^{t} component tentatively present (insufficient MRM transitions present to definitively confirm presence)
✓^{#} component not confirmed by LC-MS/MS due to low sensitivity in negative ion polarity, however a peak in the 3H-radiochromatogram indicates that component is present
X = Not determined

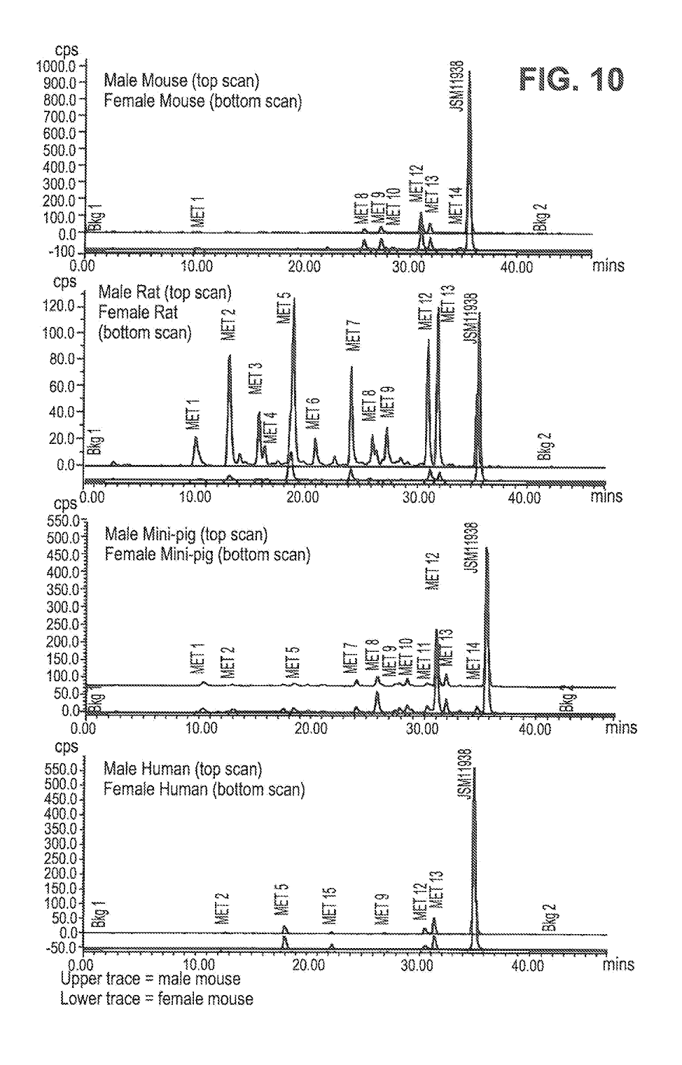

Parent ³H-CFMQ was shown to be stable in the absence of hepatocytes and the rate of metabolism of ³H-CFMQ was similar in mouse, female rat, mini-pig and human (FIG. 10). In vitro metabolism of ³H-CFMQ at 5 μM was most extensive in male rat hepatocytes, with approximately 95% of the parent metabolized by 4 hours. The rate of metabolism was markedly lower in female rat (approximately 32%) as well as both sexes of other species (mini-pig 52-57% and human 25-35%). At a higher concentration (10 uM), metabolism in the male and female mouse and human by 4 hours was 22% and 21%, respectively.

Incubations of CFMQ (HGT3711) in human hepatocytes produced one main metabolite, MET1, and was a mono-oxygenated structure which formed to levels of 10-11%, or 7-8% of parent. MET1 was also the main metabolite observed in incubations with mouse (2.0-3.7%) and mini-pig (18-20.56%) hepatocytes, and was observed in incubations with rat. Other minor metabolites were detected in human hepatocytes as well as in animal species. Numerous minor metabolite fragments were also detected in some species, however these were produced at levels below the deemed limit of accurate quantification (<1% of sample radioactivity) and therefore were not further detailed. There was no evidence for a human specific metabolite.

The 4 hour samples incubated with ³H-CFMQ (5 and 10 μM) were further analyzed by LC-MS/MS to identify metabolites. These studies demonstrated that both Phase I and Phase II metabolites were observed in all species including mono- and di-hydroxylated metabolites (MET1), acid metabolites, glucuronic acid and sulphate conjugates of Phase I metabolites of CFMQ. In addition glutathione conjugates of de-chlorinated and mono-hydroxy, de-chlorinated and a gluthathione conjugates were observed only in the mouse hepatocytes.

The major metabolites identified from each species (including mini-pig) were mono-oxidation products with smaller amounts of di-oxidation products. Metabolism by human liver hepatocytes was much less extensive than that seen in rats and mini-pigs, as was expected based on previous metabolic stability assays.

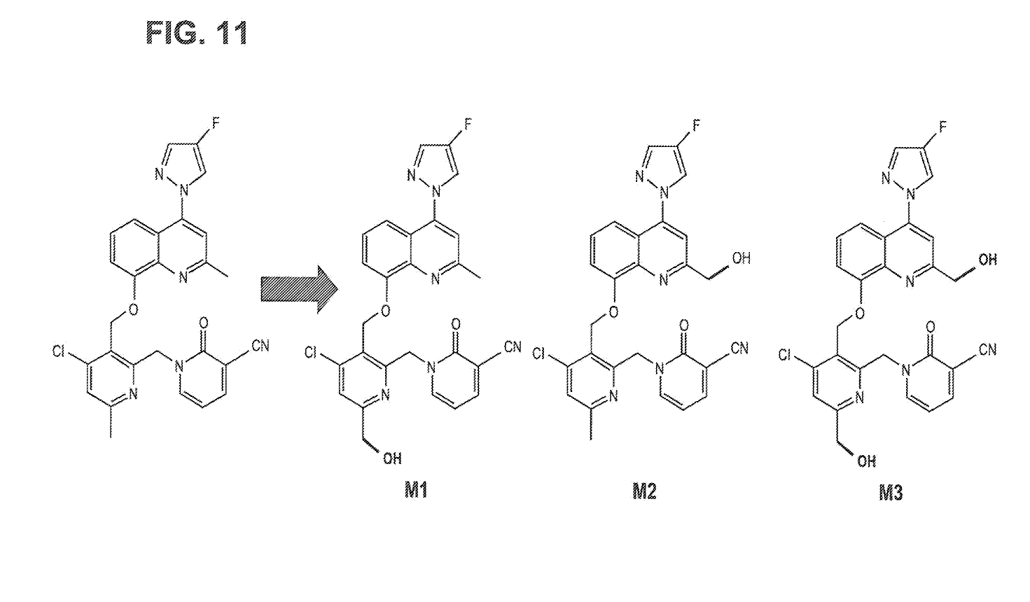

There were three major human metabolites, M1, M2 and M3 (FIG. 11). M1 was seen at 10% of parent, while M2 and M3 were formed at 5% and 3% of the parent respectively.

Results of these two studies showed that the major human hepatic metabolites of ³H-CFMQ were mono-hydroxy and di-oxygenated molecules that importantly, are produced in all other animal species examined. There may be a marked sex difference in the metabolism of CFMQ, which was also seen in the blood pharmacokinetic profile of the rat. In general the quantity of metabolites formed was greater in the nonclinical species likely due to lower stability in liver preparations. Overall, these studies suggested similar metabolism in the different species as compared to human. Importantly, each of these metabolites formed in human liver hepatocytes was also observed in the mouse and mini-pig incubations, which qualify them as a relevant nonclinical species for safety studies.

### Formulations

Various formulations of CFMQ were prepared.

A lipidic formulation of CFMQ was prepared containing 10% N-methyl pyrrolidone (NMP), 10% TRANSCUTOL HP (highly purified 2-(2-ethoxyethoxy)ethanol), 30% polyethylene glycol (PEG400), and 50% GELUCIRE 44/14 (lauroyl polyoxylglycerides). The formulation was found to give solubility, stability and exposure parameters that would allow for animal dosing experiments. Additionally, an in vivo study was conducted in Yucatan mini-pigs that demonstrated tolerability of the formulation up to 5 mL/kg. The results of that study showed that the formulation was well tolerated in mini-pigs in terms of food consumption, body weight and clinical observations.

In addition to the lipidic formulation several other approaches to formulation were taken. Three salts of CFMQ were prepared: 1,2-ethane disulfonic acid (hemi) salt, 1,5-napthalene disulfonic acid salt, and 1,2-ethane disulfonic acid (mono) salt. The 1,2ethane disulfonic (mono) salt greatly increased the solubility of CFMQ.

Additionally, CFMQ was milled to have a nano size particle.

A spray-dried dispersion approach where CFMQ was complexed with a polymer was also evaluated. When CFMQ was spray-dried onto hydroxyl propyl methyl cellulose acetate succinate (HPMCAS), the combination was found to have excellent solubility. The spray-drying process consisted of three steps: slurry preparation, spray-drying, and secondary drying. The slurry was prepared by dissolving HPMCAS polymer in a methanol/water solvent mixture (90 v/10 v), then an equivalent amount of the CFMQ was suspended in the polymer solvent mixture. The slurry was then heated and spray-dried through a flash nozzle into a nitrogen atmosphere in a spray-dryer. The powder output of the spray-dryer retained a small amount of water/methanol, which was removed in a secondary drying step, which occurred in a convection tray dryer at 40° C./15% relative humidity (RH). A 50:50 solid dispersion of the active ingredient in HPMCAS, was used in the animal studies described herein.

### Example 34

### Extrapolation of CFMQ Oral Dose by Interspecies and Pharmacokinetic Simulation

A pharmacokinetic extrapolation for a human oral dose was performed using allometric scaling of clearance and volume of distribution. The human pharmacokinetic values were extrapolated from in vivo mouse, rat, dog and monkey pharmacokinetic studies. Due to the variability in bioavailability across pre-clinical species a range of bioavailability values from 25% to 50% was modeled. This pharmacokinetic model predicted that at a human equivalent dose of 0.8 mg/kg, plasma levels will stay above the predicted efficacious levels for between greater than 5, 10 and 12 hours when bioavailability is 25, 50 or 75%. This model was based on the assumption that all of the clearance pathways in the human were captured in the pre-clinical species.

While the present teachings have been illustrated with respect to one or more implementations, alterations and/or modifications can be made to the disclosed embodiments without departing from the spirit and scope of the appended claims. In addition, while a particular feature of the present teachings can have been disclosed with respect to only one of several implementations, such feature can be combined with one or more other features of the other implementations as can be desired and advantageous for any given or particular function.

To the extent that the terms “containing,” “including,” “includes,” “having,” “has,” “with,” or variants thereof are used in either the detailed description and the claims, such terms are intended to be inclusive in a manner similar to the term “comprising.” As used herein, the term “one or more of” with respect to a listing of items such as, for example, A and B, means A alone, B alone, or A and B. The term “at least one of” is used to mean one or more of the listed items can be selected.

Notwithstanding that the numerical ranges and parameters setting forth the broad scope of the present teachings are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume values as defined earlier plus negative values, e.g., −1, −1.2, −1.89, −2, −2.5, −3, −10, −20, and −30, etc.

What is claimed is:

1. A method of treating a B₂-bradykinin receptor mediated angioedema in a subject comprising:
administering to the subject in need thereof a therapeutically effective amount of a composition comprising a compound having formula (I) or a pharmaceutically acceptable salt, stereoisomer, hydrate, or solvate thereof 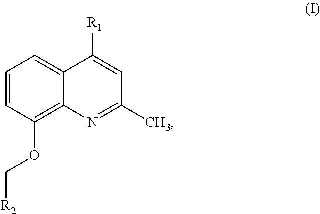
wherein R₁ is
H or 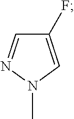
wherein R₂ is 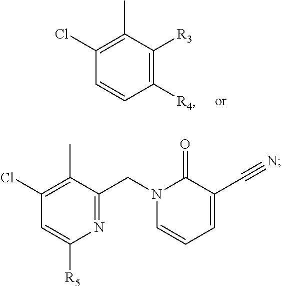
wherein R₃ is Cl or CN;
wherein R₄ is 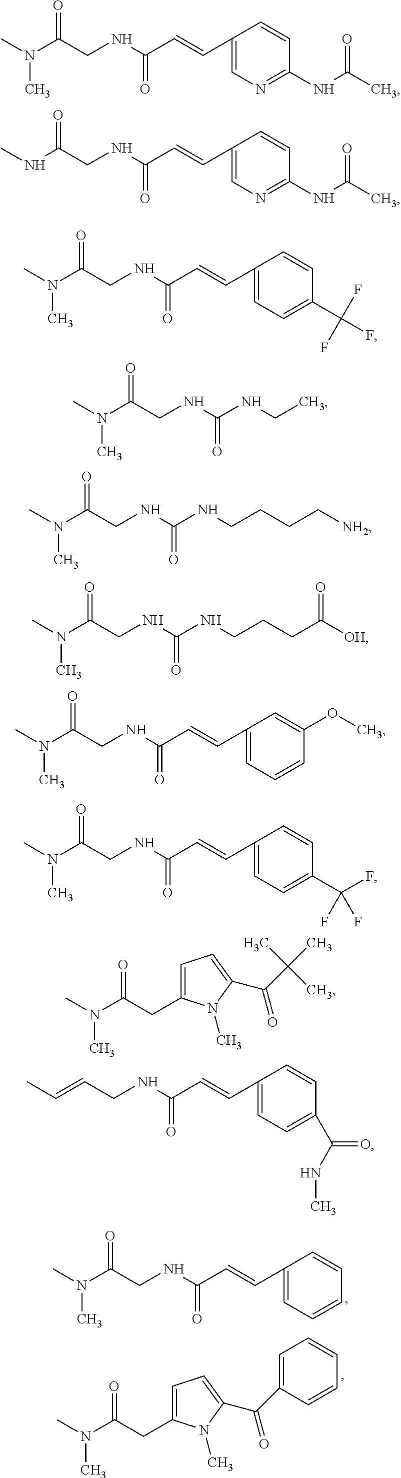 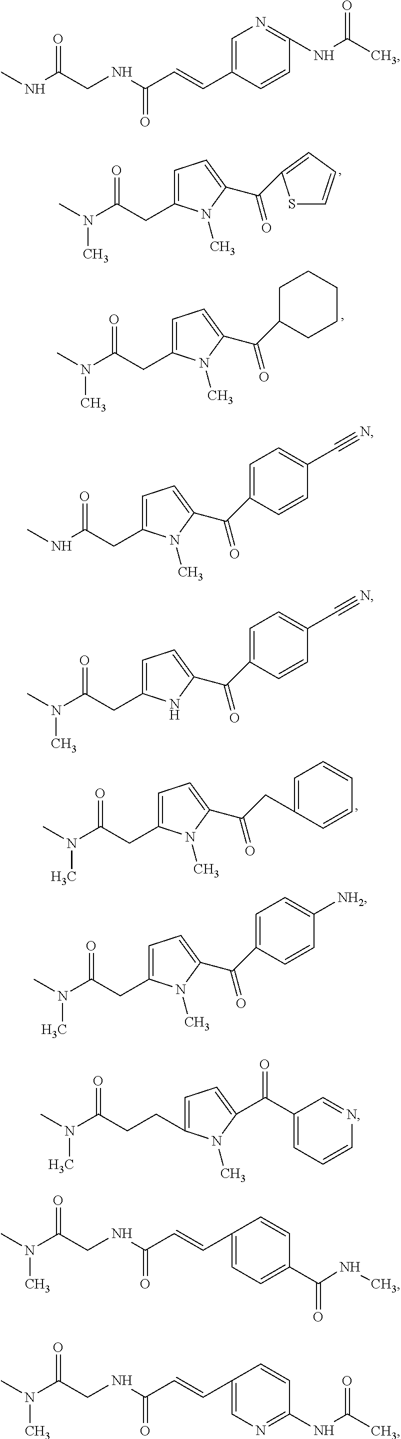 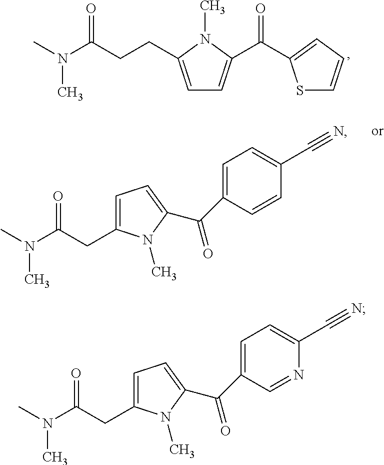
and
wherein R₅ is selected from the group consisting of H, a methyl group, an ethyl group, a propyl group, a butyl group, a pentyl group, or a hexyl group, and
wherein plasma extravasation in the subject is reduced upon administration of the compound or the pharmaceutically acceptable salt, stereoisomer, hydrate, or solvate thereof.

2. The method of claim 1, wherein the B₂-bradykinin receptor mediated angioedema is hereditary angioedema (HAE).

3. The method of claim 1, wherein the composition is administered to the subject orally or sublingually.

4. The method of claim 1, wherein the composition further comprises a pharmaceutical carrier substance, excipient, and/or an adjuvant.

5. The method of claim 1, wherein the composition is administered to the subject at about 3.0 mg of the compound having formula (I)/kg to about 35 mg of the compound having formula (I)/kg per dose and the dose is repeated within about 5 hours to about 12 hours after the initial dose.

6. The method of claim 1, wherein the method further comprises administering icatibant, ecallantide, fresh frozen plasma, C1-inhibitor, or kallikrein inhibitor to the subject.

7. The method of claim 1, wherein the compound having formula (I) has a half maximal inhibitory concentration (IC₅₀) for competition with the binding of labeled bradykinin to human B₂-bradykinin receptor of less than about 50 nanomolar.

8. The method of claim 1, wherein composition further comprises one or more of surfactants, tonicity agents, buffers, salts, preservatives, co-solvents, and viscosity building agents.

9. The method of claim 1, wherein composition in the form an aerosol, a cream, a gel, a pill, a capsule, a syrup, a solution, or a transdermal patch.

10. The method of claim 1, wherein the composition has a pH of less than about 5.

11. The method of claim 1, wherein the compound has a molecular weight less than about 650.

12. A method of treating a B₂-bradykinin receptor mediated angioedema in a subject comprising:
administering to the subject in need thereof a therapeutically effective amount a composition comprising a compound having formula (II) or a pharmaceutically acceptable salt, stereoisomer, hydrate, or solvate thereof 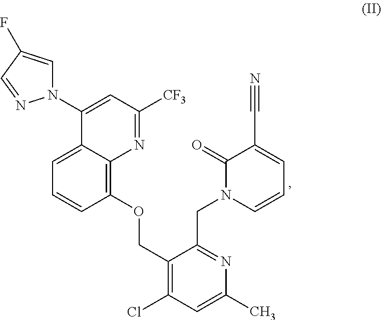 [SMILES: Cc1cc(Cl)c(COc2cccc3c(-n4cc(F)cn4)cc(C(F)(F)F)nc23)c(Cn2cccc(C#N)c2=O)n1]
thereby reducing plasma extravasation in the subject.

13. The method of claim 12, wherein the B₂-bradykinin receptor mediated angioedema is hereditary angioedema (HAE).

14. The method of claim 12, wherein the composition is administered to the subject orally or sublingually.

15. The method of claim 12, wherein the composition is administered to the subject at about 3.0 mg of the compound having formula (II)/kg to about 35 mg of the compound having formula (II)/kg per dose and the dose is repeated within about 5 hours to about 12 hours after the initial dose.

16. The method of claim 12, wherein the method further comprises administering icatibant, ecallantide, fresh frozen plasma, C1-inhibitor, or kallikrein inhibitor to the subject.

17. A method of treating a B₂-bradykinin receptor mediated angioedema in a subject comprising:
administering to the subject in need thereof a therapeutically effective amount a composition comprising
11-((4-Chloro-3-(((4-(4-fluoro-1H-pyrazol-1-yl)-2-methylquinolin-8-yl)oxy)methyl)-6-methylpyridin-2-yl)methyl)-2-oxo-1,2-dihydropyridine-3-carbonitrile;
(2E)-3-[6-(Acetylamino)pyridin-3-yl]-N-{2-[(2,4-dichloro-3-{[(2-methylquinolin-8-yl)oxy]methyl}phenyl)(methyl)amino]-2-oxoethyl}prop-2-enamide;
(2E)-3-[6-(Acetylamino)pyridin-3-yl]-N-{2-[(2,4-dichloro-3-{[(2-methylquinolin-8-yl)oxy]methyl}phenyl)amino]-2-oxoethyl}prop-2-enamide;
(2E)-N-{2-[(4-Chloro-2-cyano-3-{[(2-methylquinolin-8-yl)oxy]methyl}phenyl)(methyl)amino]-2-oxoethyl}-3-[4-(trifluoromethyl)phenyl]prop-2-enamide;
N-[4-chloro-2-cyano-3-[(2-methylquinolin-8-yl)oxymethyl]phenyl]-2-(ethylcarbamoylamino)-N-methylacetamide;
2-(4-aminobutylcarbamoylamino)-N-[4-chloro-2-cyano-3-[(2-methylquinolin-8-yl)oxymethyl]phenyl]-N-methylacetamide;
4-[[2-[4-chloro-2-cyano-N-methyl-3-[(2-methylquinolin-8-yl)oxymethyl]anilino]-2-oxoethyl]carbamoylamino]butanoic acid;
(E)-N-[2-[4-chloro-2-cyano-N-methyl-3-[(2-methylquinolin-8-yl)oxymethyl]anilino]-2-oxoethyl]-3-(3-methoxyphenyl)prop-2-enamide;
(E)-N-[2-[4-chloro-2-cyano-N-methyl-3-[(2-methylquinolin-8-yl)oxymethyl]anilino]-2-oxoethyl]-3-[4-(trifluoromethyl)phenyl]prop-2-enamide;
N-[2,4-dichloro-3-[(2-methylquinolin-8-yl)oxymethyl]phenyl]-2-[5-(2,2-dimethylpropanoyl)-1-methylpyrrol-2-yl]-N-methylacetamide;
4-[(E)-3-[[(Z)-3-[2,4-dichloro-3-[(2-methylquinolin-8-yl)oxymethyl]phenyl]prop-2-enyl]amino]-3-oxoprop-1-enyl]-N-methylbenzamide;
(E)-N-[2-[2,4-dichloro-N-methyl-3-[(2-methylquinolin-8-yl)oxymethyl]anilino]-2-oxoethyl]-3-phenylprop-2-enamide hydrochloride;
2-(5-benzoyl-1-methylpyrrol-2-yl)-N-[2,4-dichloro-3-[(2-methylquinolin-8-yl)oxymethyl]phenyl]-N-methylacetamide;
(E)-3-(6-acetamidopyridin-3-yl)-N-[2-[2,4-dichloro-3-[(2-methylquinolin-8-yl)oxymethyl]anilino]-2-oxoethyl]prop-2-enamide;
N-[2,4-dichloro-3-[(2-methylquinolin-8-yl)oxymethyl]phenyl]-N-methyl-2-[1-methyl-5-(thiophene-2-carbonyl)pyrrol-2-yl]acetamide;
2-[5-(cyclohexanecarbonyl)-1-methylpyrrol-2-yl]-N-[2,4-dichloro-3-[(2-methylquinolin-8-yl)oxymethyl]phenyl]-N-methylacetamide;
2-[5-(4-cyanobenzoyl)-1-methylpyrrol-2-yl]-N-[2,4-dichloro-3-[(2-methylquinolin-8-yl)oxymethyl]phenyl]acetamide;
2-[5-(4-cyanobenzoyl)-1H-pyrrol-2-yl]-N-[2,4-dichloro-3-[(2-methylquinolin-8-yl)oxymethyl]phenyl]-N-methylacetamide;
N-[2,4-dichloro-3-[(2-methylquinolin-8-yl)oxymethyl]phenyl]-N-methyl-2-[1-methyl-5-(2-phenylacetyl)pyrrol-2-yl]acetamide;
2-[5-(4-aminobenzoyl)-1-methylpyrrol-2-yl]-N-[2,4-dichloro-3-[(2-methylquinolin-8-yl)oxymethyl]phenyl]-N-methylacetamide;
N-[2,4-dichloro-3-[(2-methylquinolin-8-yl)oxymethyl]phenyl]-N-methyl-3-[1-methyl-5-(pyridine-3-carbonyl)pyrrol-2-yl]propanamide;
4-[(E)-3-[[2-[2,4-dichloro-N-methyl-3-[(2-methylquinolin-8-yl)oxymethyl]anilino]-2-oxoethyl]amino]-3-oxoprop-1-enyl]-N-methylbenzamide;
(E)-3-(6-acetamidopyridin-3-yl)-N-[2-[2,4-dichloro-N-methyl-3-[(2-methylquinolin-8-yl)oxymethyl]anilino]-2-oxoethyl]prop-2-enamide;
N-[2,4-dichloro-3-[(2-methylquinolin-8-yl)oxymethyl]phenyl]-N-methyl-3-[1-methyl-5-(thiophene-2-carbonyl)pyrrol-2-yl]propanamide;
2-[5-(4-cyanobenzoyl)-1-methylpyrrol-2-yl]-N-[2,4-dichloro-3-[(2-methylquinolin-8-yl)oxymethyl]phenyl]-N-methylacetamide;
2-[5-(6-cyanopyridine-3-carbonyl)-1-methylpyrrol-2-yl]-N-[2,4-dichloro-3-[(2-methylquinolin-8-yl)oxymethyl]phenyl]-N-methylacetamide;
or a pharmaceutically acceptable salt, stereoisomer, hydrate, or solvate thereof, thereby reducing plasma extravasation in the subject.

18. The method of claim 17, wherein the composition is administered to the subject orally or sublingually.

19. An oral formulation comprising a therapeutically effective amount of a compound having formula (I) or a pharmaceutically acceptable salt, stereoisomer, hydrate, or solvate thereof and a pharmaceutically acceptable carrier, wherein the therapeutically effective amount is between about 0.001 wt % and about 60 wt % of the oral formulation and formula (I) is as follows: 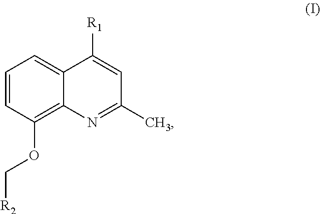
wherein R₁ is
H or 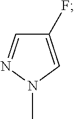
wherein R₂ is 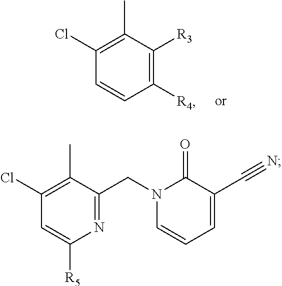
wherein R₃ is Cl or CN;
wherein R₄ is 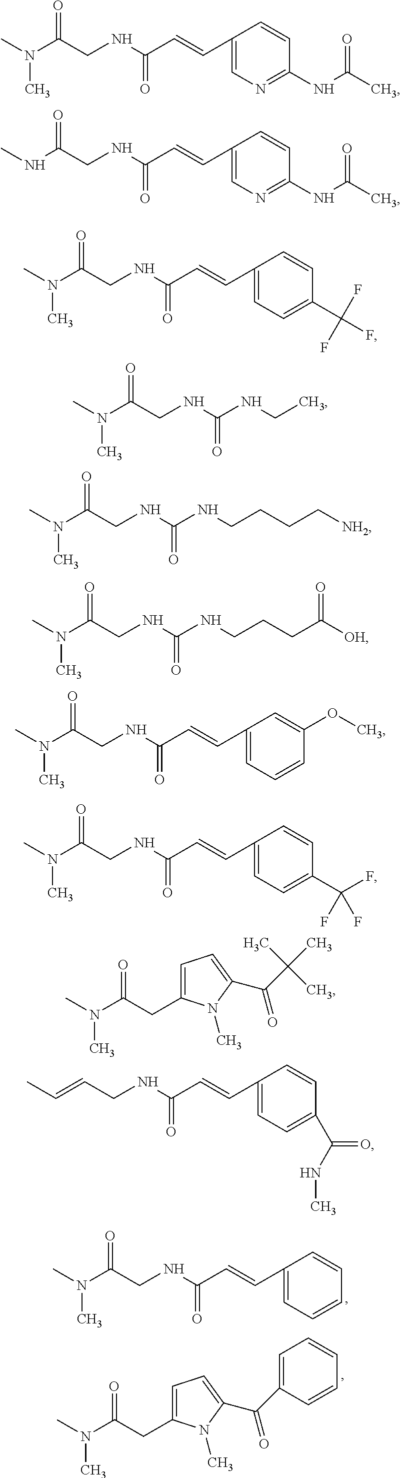 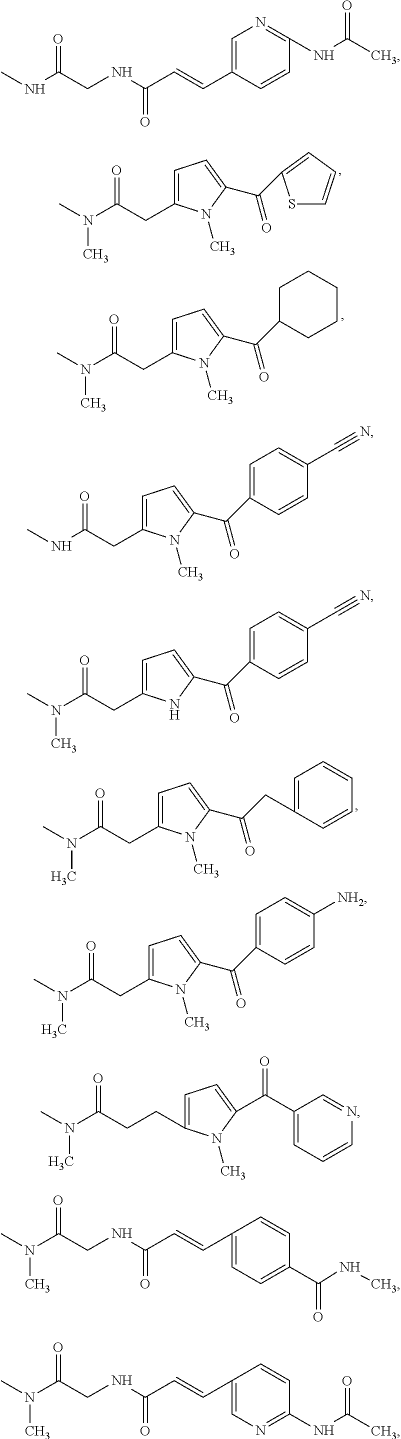 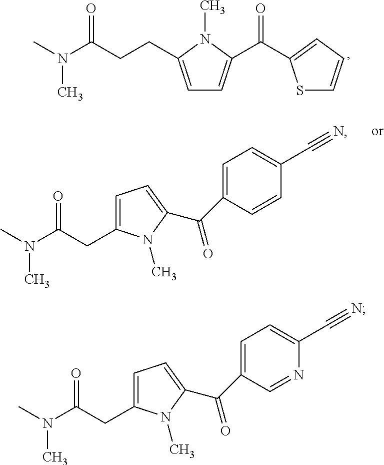
and
wherein R₅ is selected from the group consisting of H, a methyl group, an ethyl group, a propyl group, a butyl group, a pentyl group, or a hexyl group.

20. The oral formulation of claim 19, further comprising hydroxyl propyl methyl cellulose acetate succinate.

21. The oral formulation of claim 19, wherein the oral formulation is in the form of a spray-dried dispersion.

22. Use of a composition comprising a compound having formula (I) or a pharmaceutically acceptable salt, stereoisomer, hydrate, or solvate thereof for the manufacture of a medicament for the treatment and/or prevention of a B₂-bradykinin receptor mediated angioedema, wherein formula (I) is as follows: 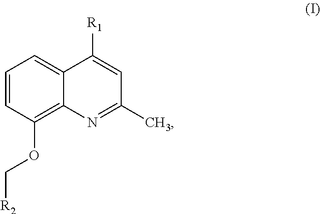
wherein R₁ is
H or 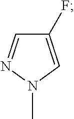
wherein R₂ is 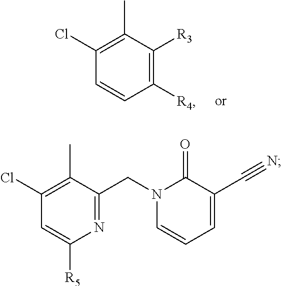
wherein R₃ is Cl or CN;
wherein R₄ is 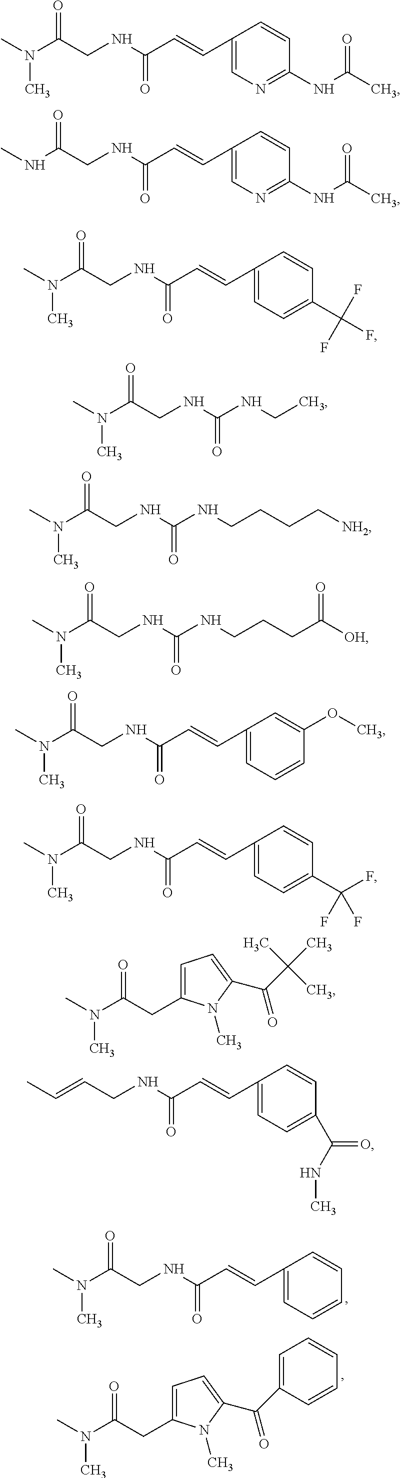 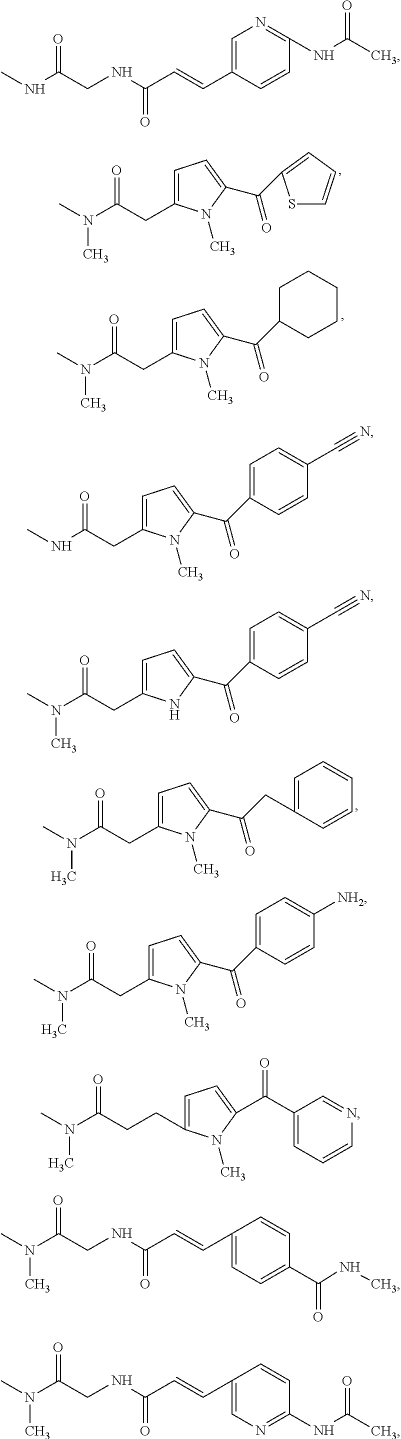 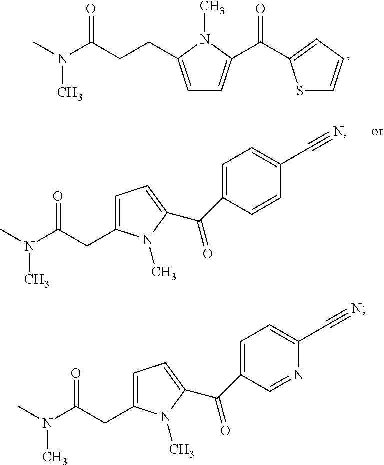
and
wherein R₅ is selected from the group consisting of H, a methyl group, an ethyl group, a propyl group, a butyl group, a pentyl group, or a hexyl group.

23. An oral formulation comprising a therapeutically effective amount of a compound having formula (II) or a pharmaceutically acceptable salt, stereoisomer, hydrate, or solvate thereof and a pharmaceutically acceptable carrier, wherein the therapeutically effective amount is between about 0.001 wt % and about 60 wt % of the oral formulation and formula (II) is as follows:

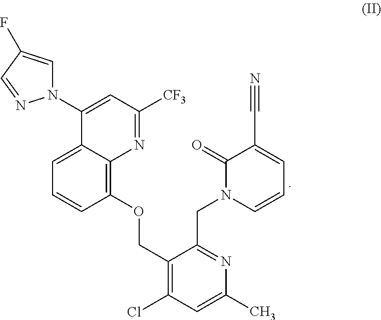 [SMILES: Cc1cc(Cl)c(COc2cccc3c(-n4cc(F)cn4)cc(C(F)(F)F)nc23)c(Cn2cccc(C#N)c2=O)n1]

24. The oral formulation of claim 23, further comprising hydroxyl propyl methyl cellulose acetate succinate.

25. The oral formulation of claim 23, wherein the oral formulation is in the form of a spray-dried dispersion.
